(19) United States
(12) Patent Application Publication
(10) Pub. No.: US 2009/0181110 A1
(43) Pub. Date: Jul. 16, 2009

(54) Compositions from Garcinia as Aromatase Inhibitors for Breast Cancer Chemoprevention and Chemotherapy

(75) Inventors: Marcy J. Balunas, Indicasat (PA); Bin Su, Glendora, CA (US); Robert W. Brueggemeier, Columbus, OH (US); Alan Douglas Kinghorn, Columbus, OH (US)

Correspondence Address: MUELLER AND SMITH, LPA;MUELLER-SMITH BUILDING, 7700 RIVERS EDGE DRIVE, COLUMBUS, OH, 43235, US

(73) Assignee: THE OHIO STATE UNIVERSITY RESEARCH FOUNDATION, Columbus, OH (US)

(21) Appl. No.: 12/169,858

(22) Filed: Jul. 9, 2008

Related U.S. Application Data
(60) Provisional application No. 60/959,448, filed on Jul. 13, 2007.

(51) Int. Cl. A61K 36/38; C12N 9/99; A61K 31/352; C12N 9/00; A61P 35/00

(52) U.S. Cl. 424/725; 435/184; 514/455; 435/4

(57) ABSTRACT

Aromatase inhibitors (AIs) are disclosed which are useful in the treatment and prevention of post-menopausal breast cancer. New AIs derived from natural products are disclosed that are evaluated for clinical utility for treating post-menopausal breast cancer and may also act as chemopreventive agents for preventing breast cancer. Several pure compounds demonstrated AI activity using a noncellular, enzyme-based microsomal and a cell-based aromatase assay. Correlations are made between structural classes with levels of aromatase inhibition. The disclosure may be utilized to direct synthetic modification of natural product scaffolds to enhance aromatase inhibition or to standardize botanical dietary supplements for increased aromatase inhibition activity.

### CROSS-REFERENCE TO RELATED APPLICATIONS

The current application claims priority based on provisional application Ser. No. 60/959,448, filed Jul. 13, 2007, the disclosure of which is expressly incorporated herein by reference.

### STATEMENT REGARDING FEDERALLY SPONSORED RESEARCH

Supported by National Cancer Institute of the National Institutes of Health grant R01 CA73698 RF #743102CI/NIH, The Ohio State University Comprehensive Cancer Center Breast Cancer Research Fund, and Chemoprevention Program.

### BACKGROUND

It is well recognized that consumption of fruits and vegetables can reduce the incidence of degenerative diseases including cancer, heart disease, inflammation, arthritis, immune system decline, brain dysfunction, and cataracts. These protective effects have been considered to be mainly to be due to the presence of various antioxidants in fruits and vegetables. Antioxidants seem to be very important in the prevention of disease because of inhibition or delay of the formation of oxidizable substrate chain reactions. Numerous investigations have indicated that free radicals cause oxidative damage to lipids, proteins, and nucleic acids. For additional information see, for example:
Gordon, M. H. Dietary antioxidants in disease prevention. Nat. Prod. Rep. 13: 265-273 (1996).
Ames, B. N.; Shigenaga, M. K.; Hagen, T. M. Oxidants, antioxidants, and the degenerative diseases of aging. Proc. Natl. Acad. Sci. U.S.A., 90: 7915-7922 (1993).

Cancer is one of the leading causes of death in adult humans. Cancers of the breast include some cancers with a particularly high incidence of morbidity. Certain females are known to be at risk for occurrence or reoccurrence of breast cancer due to genetic factors, predisposition, previous cancers, age, or hormone therapy. Certain women are prescribed drugs in the hopes of suppressing the incidence of cancers, particularly those who have had a breast cancer, or are otherwise predisposed. Unfortunately, drug therapy is fraught with undesirable side effects, and these drugs are also not entirely effective.

Of all cancers, breast cancer is the most common cancer afflicting females worldwide, with over one million incident cases, and causing nearly 400,000 deaths annually. In the United States alone, approximately 200,000 women were expected to be newly diagnosed with breast cancer in 2006, and over 40,000 deaths were predicted to occur from the disease. Estrogen hormones and their interactions with estrogen receptors (ERs) are widely recognized to play an important role in the development and progression of breast cancer. Estrogens are known to have various effects throughout the body including positive effects on the brain, bone, heart, liver, and vagina, along with negative effects such as increased risk of breast and uterine cancers with prolonged estrogen exposure. Additional information on the effects of estrogen are available from the following, along with the references cited therein:
Kuller, L. H., Matthews, K. A., and Meilahn, E. N. “Estrogens and women's health: interrelation of coronary heart disease, breast cancer and osteoporosis.” J. Steroid Biochem. Mol. Biol. 74:297-309, 2000.
Stevenson, J. C. “Cardiovascular effects of oestrogens.” J. Steroid Biochem. Mol. Biol. 74:387-393 (2000).

Modulation of estrogens and ERs can be accomplished by a number of strategies, including, by inhibiting ER binding, by downregulating ERs, or by decreasing estrogen production. FIG. 1 shows a simplified diagram of the interactions of steroid precursors, estrogens and cellular components during estrogen metabolism in a hypothetical cell. Certain compounds are known that interact with specific components of this system. Tamoxifen (Nolvadex®), is a selective estrogen receptor modulator (SERM), that works by blocking the binding of estrogen to the ER. Tamoxifen was previously considered the treatment of choice for estrogen abatement for the last twenty-five years. However, recently it has been recognized that tamoxifen acts as both an ER antagonist and agonist in various tissues, resulting in significant side-effects such as increased risk of endometrial cancer and thromboembolism. The partial antagonist/agonist activity of such compounds are also thought to lead to the development of drug resistance in certain neoplasms, leading to eventual treatment failure for patients using prophylactic and therapeutic tamoxifen.

Certain of the deleterious effects of present treatment modalities may be avoided by specifically targeting particular biochemical pathways that are involved in estrogen metabolism and modulation of cellular activities through estrogens. One such strategy is to decrease estrogen production by modulation of aromatase activity. In those women at risk of developing or being treated for estrogen dependent neoplasias, clinical agents exhibiting almost complete estrogen ablation may be indicated for certain postmenopausal women.

Aromatase is a cytochrome P450 dependent enzyme responsible for catalyzing the biosynthesis of estrogens (e.g., estrone and estradiol) from androgens (e.g., androstenedione and testosterone). The aromatase enzyme is encoded by the aromatase gene, CYP19, whose expression is regulated by tissue-specific promoters; thus, aromatase expression is apparently regulated differentially in various tissues. Aromatase expression has been identified in numerous tissues throughout the body including in tissues of the breast, skin, brain, adipose, muscle, and bone. Inhibition of the aromatase enzyme is known to reduce estrogen production throughout the body, potentially to nearly undetectable levels. Such inhibition is thought to suppress estrogen production, resulting in a significant affect on the development and progression of hormone-responsive breast cancers. Additional description of the role of aromatase may be found in:
Simpson, E. R., Zhao, Y., Agarwal, V. R., Michael, M. D., Bulun, S. E., Hinshelwood, M. M., Graham-Lorence, S., Sun, T., Fisher, C. R., Qin, K., and Mendelson, C. R., “Aromatase expression in health and disease.” Recent Prog. Horm. Res. 52:185-213 (1997).
Smith, I. E., and Dowsett, M.: Aromatase inhibitors in breast cancer. N. Engl. J. Med. 348:2431-2442 (2003).
Kendall, A., and Dowsett, M.: Novel concepts for the chemoprevention of breast cancer through aromatase inhibition. Endocr. Rel. Cancer 13:827-837 (2006).
Brueggemeier, R. W.: Update on the use of aromatase inhibitors in breast cancer. Expert Opin. Pharmacother. 7:1919-1930 (2006).

Aromatase is the rate-limiting enzyme responsible for catalyzing biosynthesis of estrogens from androgens. As shown in FIG. 1, aromatase and aromatase inhibitors may play a crucial role in controlling estrogen activated gene expression. FIG. 2 shows a number of nonsteroidal and steroidal aromatase inhibitors that are known, none of which are xanthones or analogs of xanthones. Clinically available aromatase inhibitors (AIs) have been shown to reduce estrogen production throughout the body to nearly undetectable levels, proving to have significant effects on the development and progression of hormone-responsive breast cancers. Three AIs currently in clinical use include anastrozole (Arimidex®), letrozole (Femara®), and exemestane (Aromasin®). These agents have shown nearly complete estrogen suppression and are highly selective for aromatase. When compared with currently existing breast cancer therapies, aromatase inhibitors generally exhibit significantly improved efficacy with fewer side effects. However, postmenopausal breast cancer patients eventually develop resistance to AIs, causing relapse of the disease as estrogen production recovers, which may result in tumor regrowth after 12-18 months of treatment and stable disease remission. Utilization of synthetic AIs may provide improved efficacy when used in combination treatment in order to minimize development of resistance. FIG. 3 shows a diagram of the reactions in aromatase catalyzed conversion of androgens to estrogens.

Although more recent synthetic AIs provide an improved side effect profile compared to tamoxifen, serious side effects still occur, as an effect of estrogen deprivation. Such side effects include decreased bone mineral density, osteoporosis, and increases in musculoskeletal disorders. Synthetic AIs also can result in increased negative cardiovascular events as well as altering the lipid profiles of patients. Synthetic AIs can also affect cognition, decreasing the protective effects of estrogens on memory loss with aging. Several quality of life side effects are also often seen with the use of synthetic AIs including diarrhea, vaginal dryness, diminished libido, and dyspareunia. For additional information on the side effects of presently available aromatase inhibitors, see:
Gnant, M.: Management of bone loss induced by aromatase inhibitors. Cancer Invest. 24:328-330 (2006).
Esteva, F. J., and Hortobagyi, G. N.: Comparative assessment of lipid effects of endocrine therapy for breast cancer: implications for cardiovascular disease prevention in postmenopausal women. Breast 15:301-312 (2006).
Yue, X., Lu, M., Lancaster, T., Cao, P., Honda, S., Staufenbiel, M., Harada, N., Zhong, Z., Shen, Y., and Li, R.: Brain estrogen deficiency accelerates Abeta plaque formation in an Alzheimer's disease animal model. Proc. Natl. Acad. Sci. U.S.A. 102:19198-19203 (2005).

With the clinical success of several synthetic AIs for the treatment of post-menopausal breast cancer, researchers have begun investigating the potential of natural products as AIs. For example, Phase I clinical trials have recently begun on the botanical dietary supplement IH636 grape seed extract for the prevention of breast cancer in postmenopausal women who are at increased risk of developing breast cancer. See also for example, U.S. Patent Publication No. 2004156926 by Anderson, entitled, “Inhibiting aromatase with specific dietary supplements.” For additional information, see:
Eng, E. T., Ye, J., Williams, D., Phung, S., Moore, R. E., Young, M. K., Gruntmanis, U., Braunstein, G., and Chen, S.: Suppression of estrogen biosynthesis by procyanidin dimers in red wine and grape seeds. Cancer Res. 63:8516-8522 (2003).
Kijima, I., Phung, S., Hur, G., Kwok, S. L., and Chen, S.: Grape seed extract is an aromatase inhibitor and a suppressor of aromatase expression. Cancer Res. 66:5960-5967 (2006).
Moongkamdi, P, N, Kosem, S Kaslungka, 0 Luanratana, N Pongpan, and N Neungton. Antiproliferation, antioxidation and induction of apoptosis by Garcinia mangostana (mangosteen) on SKBR3 human breast cancer cell line. 1. Ethnopharmacol. 90, 161-166 (2004).

Consumption of fruits and vegetables have also recently been related to chemoprevention of cancer. Cancer chemoprevention refers to intervention such as the prevention, delay or reversal of the process of carcinogenesis by the ingestion of either naturally occurring or synthetic dietary constituents, including food, dietary supplements, drugs or synthetic agents in order to limit cancer initiation and progression. Of the various processes of carcinogenesis, blocking of tumor initiation by carcinogens is considered an important step in protecting cells through the induction of Phase II drug-metabolizing enzymes such as glutathione-S-transferase and quinone reductase. See:
Talalay, P., Fahey, J. W., Holtzclaw, W. D., Prestera, T., Zhang, Y., Chemoprotection against cancer by phase 2 enzyme induction. Toxicol. Lett. 82-83, 173-179 (1995).

For instance, fruits and vegetables contain many identifiable chemopreventive agents, including for instance, carotenoids, flavanoids and antioxidants. Fruit products are thus widely recognized in the food science art as a source of a number of health promoting phytochemicals. (Johns et al., Recent Advances in Phytochemistry, pp. 31-52, Plenum Press (1997)).

The metabolism of carcinogens and the detoxification of carcinogenic compounds is subject to active study, and the control of these processes is important for chemotherapy and chemopreventive treatments. Moreover, chemopreventive compounds may be useful for modulating cellular metabolism to prevent or impede the initiation and progression of cancers.

In certain instances, consumption of fresh or preserved fruits and vegetables may be effective for providing a chemopreventive benefit. More commonly, beneficial substances present in fruits and vegetables are present in very small concentrations in the food. Providing for the addition of substances derived from fruits and vegetables in therapeutically effective concentrations would allow for the consumption of beneficial chemopreventive substances without excessively increasing the calorie content or volume of food consumed. Thus, in light of the known correlations between diet and incidence of cancer, there is a need to provide dietary supplements that deliver beneficial phytochemicals at concentrations sufficient to modulate cell dysplasia, inhibit neoplasias, reduce cancer incidence and inhibit the progression of precancerous lesions to cancer.

Garcinia mangostana L. (Clusiaceae), commonly known as mangosteen, is referred to as “the queen of fruits” in Thailand and is a slow-growing tropical evergreen tree with leathery, glabrous leaves attaining 25 m in height. Mangosteen has dark purple to red-purple fruits. The edible fruit aril is white, soft, and juicy with a sweet, slightly acid taste. The fruit hull of G. mangostana has been used as a traditional medicine in Southeast Asia for the treatment of skin infections, diarrhea, inflammation, wounds, and ulcers. Recently, products manufactured from G. mangostana have begun to be used as a botanical dietary supplement in the United States, because of their potent antioxidant potential. The major secondary metabolites of mangosteen have been found to be prenylated xanthone derivatives. Some members of this compound class isolated from mangosteen have been associated with a variety of antifungal, antimicrobial, antioxidant, and cytotoxic activities. Prenylated xanthone derivatives are not widely produced in plants, but are found in members of the genus Garcinia, among other related plants. See also:
Farnsworth, N. R., Bunyapraphatsara, N. 1992. Thai Medicinal Plants Recommended for Primary Health Care System. Prachchon Co., Bangkok, pp. 160-162.
Peres, V., Nagem, T. J., de Oliveira, F. F., Tetraoxygenated naturally occurring xanthones. Phytochemistry 55, 683-710 (2000).
Suksamrarn, S., Komutib, O., Ratananukul, P., Chimnol, N., Lartpornmatulee, N., Sukamrarn, A., Cytotoxic prenylated xanthones from the young fruit of Garcinia mangostana. Chem. Pharm. Bull. 54, 301-305 (2006)
Garrity, A. R., Morton, G. A., Morton, J. C., 2004. Nutraceutical mangosteen composition. U.S. Pat. No. 6,730,333 B1 20040504.

The most abundant xanthone from G. mangostana, α-mangostin, was found to inhibit alveolar duct formation in a mouse mammary organ culture model and to suppress the carcinogen-induced formation of aberrant crypt foci in a short-term colon carcinogenesis model. The potential cancer chemopreventive activity of G. mangostana extracts is, thus, suggested, but there have been no report on the ability of the G. mangostana xanthones to inhibit aramatase.

Moreover the nature of the composition of the complex xanthones from mangosteen extracts is not previously known. Certain mangosteen preparations on the market are standardized to a given concentration of α-mangostin. While mangosteen preparations may provide a therapeutic and or chemopreventive benefit, standardization of the extract preparations to a given concentration of a biochemically significant compound would be advantageous, rather than to simply standardize to the most prevalent compound.

In light of the apparent benefits provided by aromatase inhibitors, along with the negative side effects associated with presently available compounds, there exists a continuing need for additional and improved AIs with an more beneficial side effect profile.

### SUMMARY OF THE INVENTION

The present disclosure generally relates to preparations and compositions of natural and or synthetic xanthones that provide a chemotherapeutic benefit. The disclosure is further embodied more particularly as a derivative from mangosteen useful for disease prevention and therapy. In addition, other related compounds from licorice are disclosed.

One embodiment is a method of inhibiting aromatase activity comprising providing a composition of matter consisting essentially of an extract of mangosteen therapeutically effective for inhibiting aromatase activity.

A further, preferred embodiment is a method of inhibiting aromatase activity comprising a providing a xanthone compound with aromatase inhibiting activity represented by Formula I:

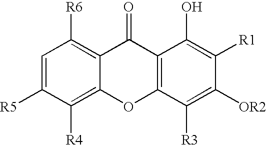

wherein:
R1 is a prenyl group or a hydrocarbon of five or more carbons or esters thereof;
R2 is —H, —OH, —CH₃ or a hydrocarbon or esters thereof;
R3 is —H, a prenyl group, or a hydrocarbon of five or more carbons or esters thereof;
R4 is —H, —OH, —OCH3, a prenyl group or a hydrocarbon of five or more carbons or esters thereof; and
R5 is —H, or —OH;
R6 is —H, —OH, —OCH3, a prenyl group, a hydrocarbon of five or more carbons, a hydroxlyated hydrocarbon of 5 carbons or more, or esters thereof; and pharmaceutically acceptable salts thereof.

In an even more preferred embodiment, R1 is a prenyl group, R2 is an —H, R3 is an —H, R4 is an —H, R5 is an —OH, and R6 is a prenyl group or a 5 carbon hydroxylated group. As such, the method comprises compounds wherein the compound is one or more of garcinone D and garcinone E, 1-isomangostin, mangostinone, α-mangostin, and γ-mangostin. Furthermore, while using the method, the compound may be administered to a subject patient as a foodstuff, dietary supplement or pharmaceutical composition and or drug fortified with a xanthone according to Compound 1 or analog thereof having a therapeutically effective amount of activity in modulating undesired signal transduction activity useful for reducing the frequency, duration or severity of a disease or condition in a subject. Such a subject in need of therapy would include a subject who has, or is at elevated risk for acquiring a malignancy, in particular, wherein the subject has, has had, or is at elevated risk of developing breast cancer or other estrogen sensitive disease.

In yet another embodiment, a method is provided for standardizing a nutraceutical product comprising identifying a xanthone from mangosteen with significant aromatase inhibiting ability to function as a marker compound; measuring the amount of said xanthone in the ingredients for said nutraceutical product; and adjusting the composition of said nutraceutical product by the addition of a given amount of said xanthone or inert ingredient wherein the standardized a nutraceutical product contains an identified concentration of said xanthone. The method of standardizing may utilize xanthones with identifiable chemotherapeutic benefit, wherein the nutraceutical product is standardized to provide a given amount per dose of xanthone of one or more of cudraxanthone G, 8-deoxygartanin, garcinone D, garcinone E, gartanin, 8-hydroxycudraxanthone G, 1-isomangostin, α-mangostin, γ-mangostin, mangostinone, smeathxanthone A, and tovophylline A. Of particular value is standardization to a quantity of an aromatase inhibitor such as garcinone D, garcinone E, α-mangostin, and γ-mangostin.

Disease may be treated by providing a composition comprising an extract having a therapeutically effective amount of activity in modulating undesired signal transduction activity useful for reducing the frequency, duration or severity of a neoplastic disease or condition in a subject, said extract being derived from a plant of the genus Garcinia. Diseases believed to be amenable to treatment as described include, diseases or conditions selected from the group consisting of a malignancy, a neoplasia, an inflammatory disease or condition, an immunological disease, or aging, and in particular breast cancer.

In certain preferred embodiments, the composition is obtained from the pericarp of mangosteen. As such, the composition possesses an amount of activity useful for modulating undesired signal transduction activity at least about 100% greater than present in the juice of mangosteen pericarp. The composition is preferably provided in a form suitable for use in one or more of a foodstuff, a dietary supplement, a drug and a pharmaceutical composition, along with suitable carriers therefore.

Furthermore a method is provided for treating or preventing a disease or condition in a subject comprising the step of administering to said subject a therapeutically-effective amount of a foodstuff, dietary supplement or pharmaceutical composition fortified with a xanthone according to Compound 1 or analog thereof having a therapeutically effective amount of activity in modulating undesired signal transduction activity useful for reducing the frequency, duration or severity of a disease or condition in a subject. In this method, the disease or condition may be selected from the group consisting of a malignancy, an immunological disease, aging or breast cancer. In addition, the xanthone is provided to a subject who has, or is at elevated risk for acquiring a malignancy, with such xanthone being one or more of cudraxanthone G, 8-deoxygartanin, garcinone D, garcinone E, gartanin, 8-hydroxycudraxanthone G, 1-isomangostin, α-mangostin, γ-mangostin, mangostinone, smeathxanthone A, and tovophylline A.

### BRIEF DESCRIPTION OF THE DRAWINGS

For a fuller understanding of the nature and advantages of the present disclosure, reference should be had to the following detailed description taken in connection with the accompanying drawings, in which:

FIG. 1 shows a diagram of the interactions of aromatase in estrogen metabolism in a hypothetical cell;

FIG. 2 shows structures of aromatase inhibiting compounds;

FIG. 3 shows a diagram of the reactions in aromatase catalyzed conversion of androgens to estrogens;

FIG. 4 shows structures of compounds isolated from the pericarp of G. mangostana

FIG. 5 shows identified structures of compounds from Garcinia mangostana;

FIG. 6 shows selected HMBC correlations of compounds 1 and 2 of FIG. 1;

FIG. 7 shows a graph of the percent control activity (PCA) of extracts and compounds in a noncellular, enzyme-based, microsomal aromatase bioassay;

FIG. 8 shows IC50 curves for active compounds from mangosteen: (A) garcinone D; (B) garcinone E; (C) α-mangostin; and (D) γ-mangostin;

FIG. 9 shows percent control activity of various compositions from mangosteen in a SK-BR-3 cell-based aromatase bioassay;

FIG. 10 shows the percent cell survival following treatment with compositions from mangosteen in a SK-BR-3 cell-based cytotoxicity bioassay;

FIG. 11 shows IC50 curves for γ-mangostin in (A) SK-BR-3 aromatase bioassay and (B) SK-BR-3 cytotoxicity bioassay;

FIG. 12 shows the structures of various compounds tested from licorice (Glycyrrhiza glabra L.); and

FIG. 13 shows aromatase bioassay results for licorice extracts and compounds;

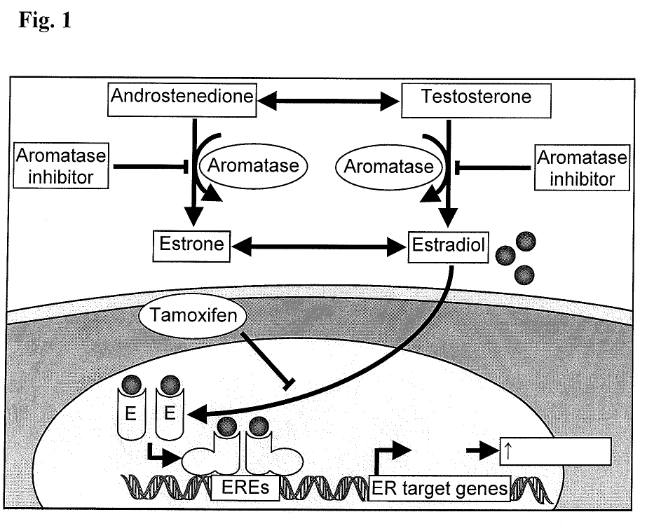

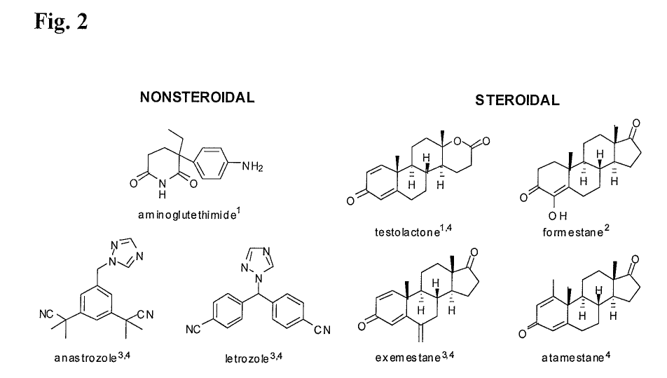

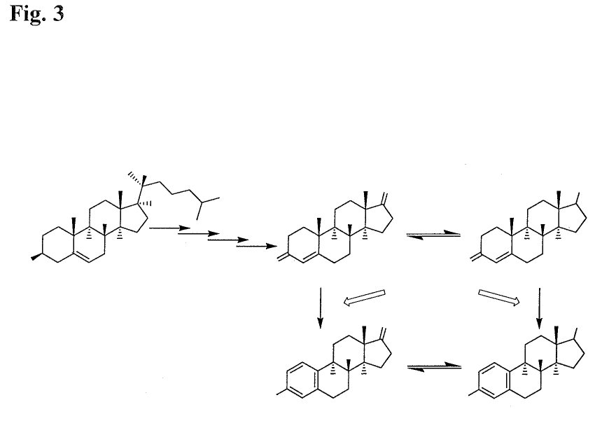

### DETAILED DESCRIPTION

The invention generally relates to a class of compounds first identified from mangosteen. Certain of the xanthones purified from mangosteen are shown herein to possess aromatase inhibitor activity.

In one embodiment, the compositions disclosed and proposed herein (particularly those that possess aromatase inhibition) can be administered to a human or other animal to treat or prevent a variety of cancers. In particular, the extracts of the invention are especially well-suited for inhibiting the development of cancers stimulated by estrogen or other steroids. A further embodiment is that even the unpurified components of the mangosteen extracts are believed to be safe for human consumption, being derived from a consumable foodstuff using consumable extraction solvents and preparations from mangosteen have been widely utilized for decades. Though xanthones are commonly present in mangosteen extracts, prior to the present disclosure, it has not been known what bioactivity these xanthones may deliver, nor which xanthones are particularly suited for delivering beneficial activity.

A further embodiment is in the modulation of specific cellular metabolic activity by the extracts and compounds disclosed herein. As such, a method is provided through which to treat cellular dysplasia, moderate the effects of neoplastic lesions and provide for a direct or adjunctive therapy for the treatment of cancer. The extracts disclosed are shown by the detailed data provided herein to possess the capability of directly or indirectly modulating the activity of specific enzymes, for instance, aromatase, and modulating the production or accumulation of signaling molecules such as estrogen and associated receptors and kinases. In the discourse that follows, the nature and effects of these beneficial activities of the extracts of the invention are further explained.

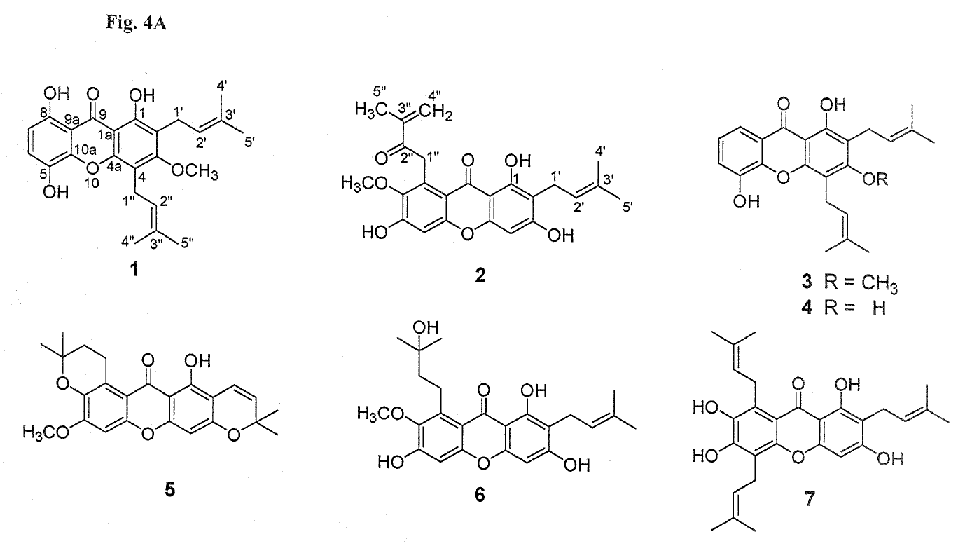

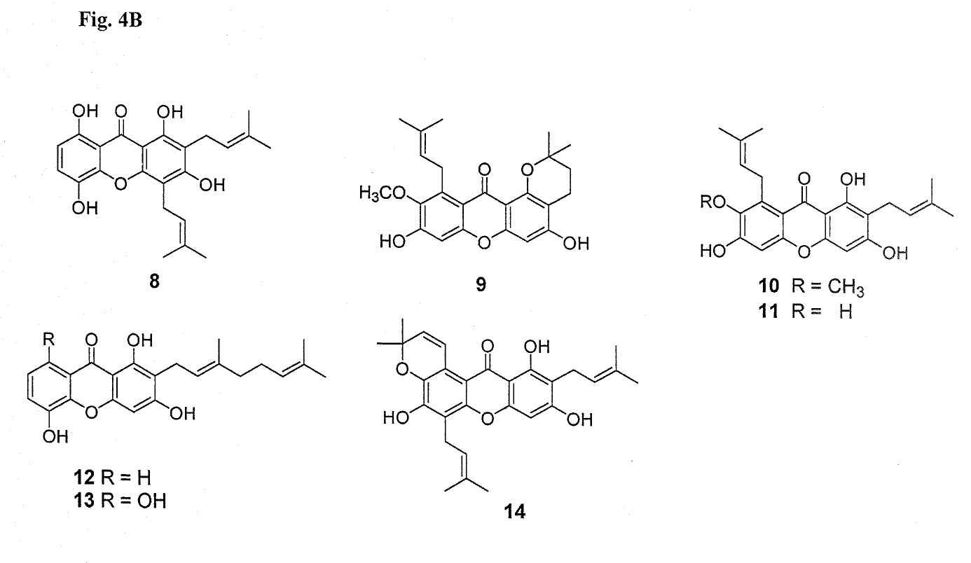

Research on the chemical constituents of mangosteen fruits provided a CH₂Cl₂-soluble partition of the MeOH extract of the pericarp of mangosteen that was found to have significant antioxidant activity in a peroxynitrite-scavenging bioassay. This extract was purified by repeated chromatography. From the fractionated extracts, two highly oxygenated prenylated xanthones were isolated. In addition, several other xanthone compounds were further characterized. As shown in FIG. 4, the compounds 8-hydroxycudraxanthone G (1) and mangostingone (2), were identified as well as 12 previously characterized xanthones. These compounds were isolated in a form that was amenable to analysis in a mouse mammary organ culture ex vivo assay. For additional information see:
Jung, H-A, Su, B.-N., Keller, W. J, Mehta, R. G., and Kinghorn A. D., “Antioxidant Xanthones from the Pericarp of Garcinia mangostana (Mangosteen) J. Agric. Food Chem. 54: 2077-2082 (2006).

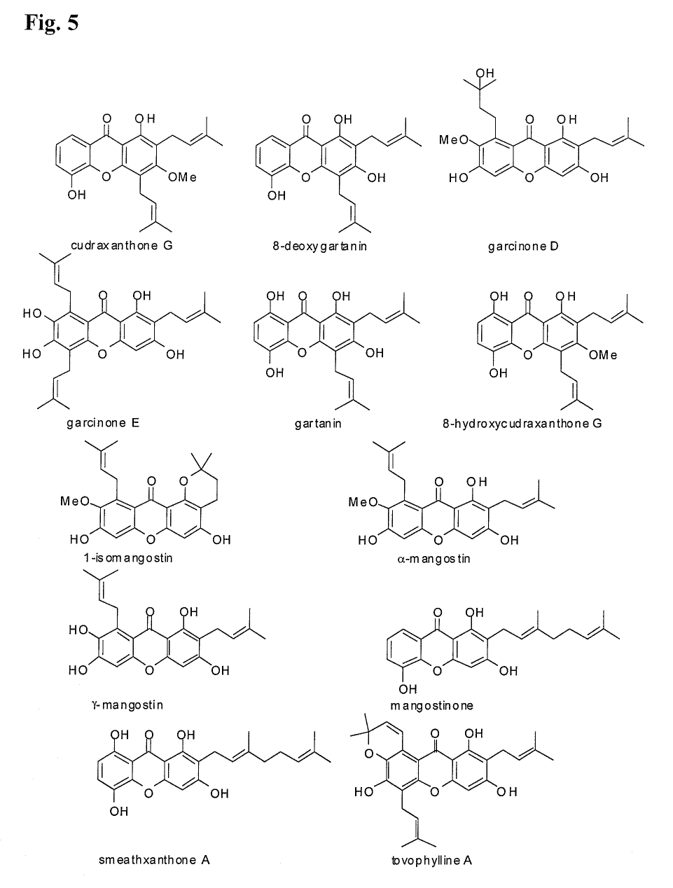

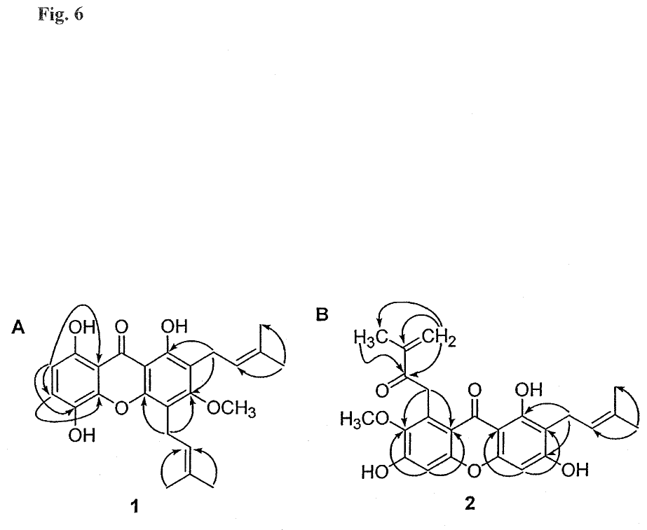

Repeated column chromatography of the CH₂Cl₂-soluble fraction of the pericarp of G. mangostana led to the isolation of two newly identified compounds (1 and 2) along with 12 previously characterized prenylated xanthones (See FIG. 4). The structures of the known compounds cudraxanthone G (3), 8-deoxygartanin (4), garcimangosone B (5), garcinone D (6), garcinone E (7), gartanin (8), 1-isomangostin (9), α-mangostin (10), γ-mangostin (11), mangostinone (12), smeathxanthone A (13), and tovophyllin A (14) were identified by comparing their physical and spectroscopic data (UV, 1H NMR, 13 C NMR, DEPT, and 2D NMR) with those of published values and were confirmed by their HRESIMS data. Additional representations of the structure of xanthones from mangosteen are shown in FIG. 5.

Compounds 10 and 11, a-mangostin, γ-mangostin, respectively, were found to be the major components of the CH₂Cl₂-soluble extract of the pericarp of G. mangostana.

New cancer chemopreventive agents from the fruits of Garcinia mangostana L. (Clusiaceae) (mangosteen) were identified for further investigation when a dichloromethane-soluble extract of these fruits was found to exhibit inducing activity of quinone reductase (QR) in cultured murine hepatoma cells (Hepa 1c1c7). See the Examples for further discussion.

Bioactivity-guided fractionation of a dichloromethane-soluble extract of Garcinia mangostana fruits was used to isolate and identify five compounds, as shown in FIGS. 4 and 5, including two xanthones, 1,2-dihydro-1,8,10-trihydroxy-2-(2-hydroxypropan-2-yl)-9-(3-methylbut-2-enyl)furo[3,2-a]xanthen-11-one and 6-deoxy-7-demethylmangostanin, along with three other known compounds, 1,3,7-trihydroxy-2,8-di-(3-methylbut-2-enyl)xanthone, mangostanin, and α-mangostin. The structures of the new compounds were determined from their spectroscopic data. These isolated compounds together with eleven other compounds previously isolated from the pericarp of mangosteen, were tested in the in vitro quinone reductase-induction assay using murine hepatoma cells (Hepa 1c1c7) and an in vitro hydroxyl radical antioxidant assay. Of these, compounds 4 induced quinone reductase (concentration to double enzyme induction, 0.68-2.21 μg/mL) in Hepa 1c1c7 cells and γ-mangostin exhibited hydroxyl radical-scavenging activity (IC50, 0.20 μg/mL). Research on the chemical constituents of mangosteen fruits provided a CH₂Cl₂-soluble partition of the MeOH extract of the pericarp of mangosteen that was found to have significant antioxidant activity in a peroxynitrite-scavenging bioassay. This extract was later purified by repeated chromatography, and followed by the isolation of two highly oxygenated prenylated xanthones. As shown in FIG. 4, the compounds 8-hydroxycudraxanthone G (1) and mangostingone (2), were identified as well as 12 better characterized xanthones. The ability of these compounds to function as antioxidants, as measured by an peroxynitrile scavenging assay is shown in Table 1.

TABLE 1
Peroxynitrite Scavenging Activity of Compounds Isolated
from the Pericarp of G. Mangostana (Mangosteen)

Peroxynitrite

| | Authentic | SIN-1-derived |
|---|---|---|
| | ONOO⁻ | ONOO⁻ |
| COMPOUND | (_{IC50}, μM) | (_{IC50}, μM) |
| 1 (8-hydroxycudraxanthone G) | 4.6 | 10.0 |
| 2^{a} (mangostingone) | — | — |
| 3 (cudraxanthone G) | >30 | 3.2 |
| 4 (8-deoxygartanin) | >30 | 11.9 |
| 5 (garcimangosone B) | 15.9 | >30 |
| 6 (garcinone D) | 26.4 | 15.1 |
| 7 (garcinone E) | 14.1 | <30 |
| 8 (gartanin) | 9.1 | 9.3 |
| 9 (1-isomangostin) | 19.2 | 24.1 |
| 10 (a-mangostin) | 12.2 | >.49 |
| 11 (γ-mangostin) | 8.0 | 3.1 |
| 12 (mangostinone) | >30 | >30 |
| 13 (smeathxanthone A) | 2.2 | 9.7 |
| 14 (tovophyllin A) | >30 | >30 |
| DL-pencillamine^{b} | 3.1 | 7.4 |

^{a}Compound 2 was not evaluated in these assays because it was isolated in insufficient quantity.
^{b}Positive control. A compound is considered to be inactive if its _{IC50} value is >30 μM

The antioxidant activities of 13 isolated compounds (1 and 3-14) were determined using the authentic ONOO— and SIN— 1-derived ONOO— methods. Compound 2 was initially obtained in insufficient amounts for this testing. The scavenging activities on ONOO— of the compounds tested are as summarized above in Table 1.

Five of the xanthones (1, 8, 10, 11, and 13) were demonstrated to possess potent antioxidant activity in both assays tested. The species ONOO—, generated from NO. and O2.— in vivo, has been reported to act as an oxidant and be involved in the initiation of carcinogenesis, along with NO. Because there is a lack of defense systems against ONOO— in the body and the highly reactive peroxynitrous acid (ONOOH), formed by protonation of ONOO—, easily decomposes to induce more highly reactive oxygen species, such as .OH, there is considerable interest in the development of ONOO— scavengers. Until now, two possible pathways of phenolic compounds to scavenge ONOO— may be represented by nitration and electron donation. Monohydroxylated phenolic compounds, such as ferulic acid and p-coumaric acid, act as ONOO— scavengers by nitration. On the other hand, compounds with a catechol moiety, such as caffeic acid and chlorogenic acid, reduce ONOO— generated from NO. and O2.— by electron donation. The presence of two hydroxyl groups at the C-5 and C-8 positions in compounds 1, 8, and 13 was consistent with their potent antioxidant effects (37, 38). Compounds 10 and 11 both possess hydroxyl groups at positions C-1, C-3, and C-6. These results support the use of the pericarp of G. mangostana as an antioxidant botanical dietary supplement. It is worth noting that two of the active isolates obtained in the present investigation, α-mangostin (10) and γ-mangostin (11), were found to be major components of the CH₂Cl₂-soluble extract of the pericarp of G. mangostana. Therefore, these two compounds may be used as marker components for quality control of botanical dietary supplements, nutraceutical preparations and pharmaceutical preparations derived from Garcinia.

α-Mangostin (10) and γ-mangostin (11) were evaluated for their potential to inhibit DMBA-induced preneoplastic lesions in a mouse mammary organ culture (MMOC) assay. At a concentration of 10 μg/mL, the percent inhibitions of compounds 10 and 11 were 57.1 and 42.9, respectively. The more active compound, α-mangostin (10), was then further evaluated in a dose-response MMOC assay, and it exhibited an IC₅₀ of 1.0 μg/mL (2.44 μM). Substances active in this cell based model system are considered to be good candidates for further investigation in full-term cancer chemopreventive studies in experimental animal models. In recent work, a crude α-mangostin (10) preparation was found to have efficacy in inhibiting preneoplastic lesions in a rat colon carcinogenesis model, although the basis of this activity was then unknown. Accordingly, the further investigation of extracts of magosteen pericarp and α-mangostin as potential cancer chemopreventive agents was undertaken.

Aromatase inhibitors are recognized as a beneficial agent for the prevention and treatment of a number of diseases caused by hormones, namely estrogen dependent processes. Natural products that have been used traditionally for nutritional or medicinal purposes (for example, botanical dietary supplements and ethnobotanically utilized species), and thus may provide AIs with reduced side effects. Reduced side effects may be the result of compounds within the natural product matrix that inhibit aromatase while other compounds within the matrix alleviate some of the side effects of estrogen deprivation (e.g., phytoestrogens). As such, natural product AIs are important for the translation of AIs from their current clinical uses as chemotherapy agents to future clinical uses in breast cancer chemoprevention. New natural product AIs may be clinically useful for treating postmenopausal breast cancer and may also act as chemopreventive agents for preventing breast cancer.

Extracts and pure compounds from mangosteen were screened using a noncellular, enzyme-based microsomal aromatase assay. After initial analysis, several extracts and xanthones isolated from mangosteen were found to have potent aromatase inhibition in a noncellular aromatase assay, exhibiting dose-dependent inhibition. Active compounds were further screened in a cell-based aromatase bioassay, using SK-BR-3 hormone-independent breast cancer cells that overexpress aromatase. Several extracts and xanthones isolated from mangosteen were found to have potent aromatase inhibition in the noncellular aromatase assay, exhibiting dose-dependent inhibition. Testing for activity of twelve xanthones, as isolated from G. mangostana by Jung et al., 2006, for aromatase inhibition was conducted in microsomes. Compounds from G. mangostana are shown in FIG. 5.

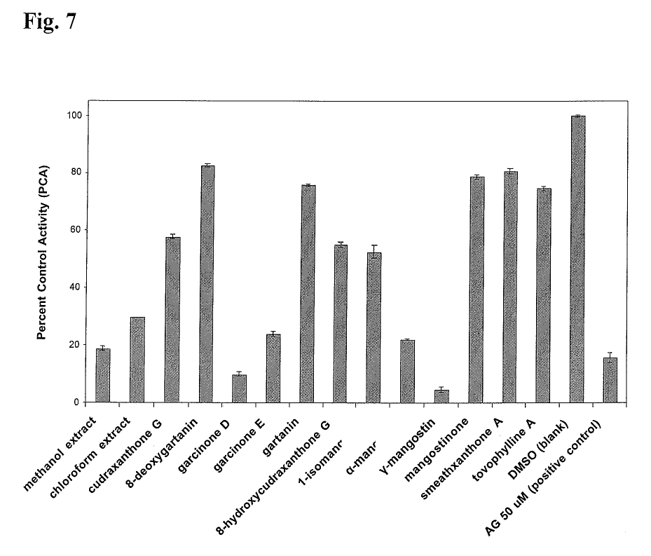

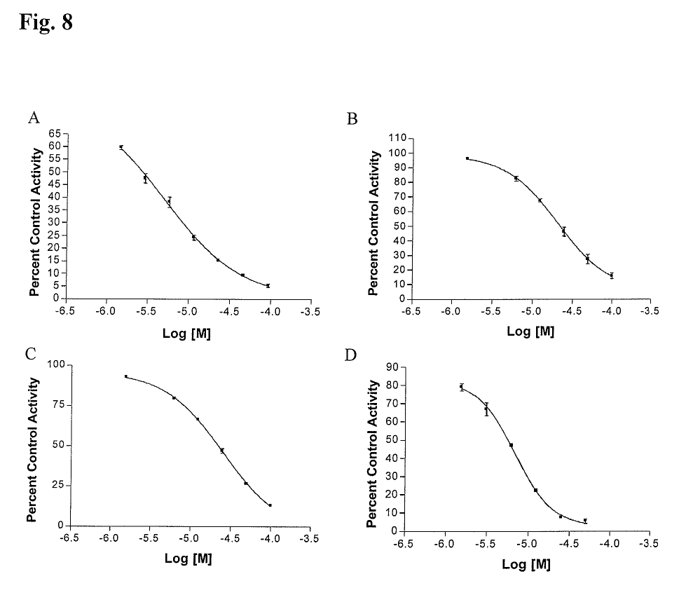

To further elucidate the biological activity of these preparations, methanol and chloroform-soluble extracts of G. mangostana fruit were tested for aromatase inhibitory activity utilizing a microsomal activity study. Certain of these compounds were found to be strongly inhibitory against aromatase in the microsomal assay. FIG. 8 shows the Percent control activity (PCA described in Examples, below) of extracts and compounds from mangosteen tested in a noncellular, enzyme-based, microsomal aromatase bioassay. As shown in FIG. 7, DMSO represents a dimethylsulfoxide, blank/negative control; AG represents a aminoglutethimide, positive control, and the remaining compounds, as identified represent the compounds listed in FIG. 5. (see also, Table 2). Active compounds were then subjected to IC50 testing to determine if they acted in a dose-dependent manner. As shown in FIG. 8, IC50 curves for active compounds from mangosteen: (A) garcinone D (IC50=5.16 μM), (B) garcinone E (IC50=25.14 μM), (C) α-mangostin (IC50=20.66 μM), and (D) γ-mangostin (IC50=6.88 μM).

Two xanthones, γ-mangostin [4.7 PCA IC50 6.9 μM] and garcinone D (10.0 PCA, IC50 5.2 μM), were found to be strongly active in microsomes (Table 2, FIGS. 7 and 8). Two other xanthones, α-mangostin (22.2 PCA, IC50 20.7 μM) and garcinone E (23.9 PCA, IC50 25.1 μM), were found to be moderately active in microsomes. All other xanthones tested were not identified as being active as provided. The compounds were arbitrarily designated as strongly active if their percent control activity (PCA) was 0-10, moderately active if their PCA was >10-30, weakly active if their PCA was 30-50, and inactive if their PCA was greater than 50.) These identified xanthones are thus among the most potent aromatase inhibitors from natural products known to date as identified using the microsomal aromatase assay.

TABLE 2
Percent control activity (PCA) of extracts and compounds from Garcinia mangostana
L. (mangosteen) in a noncellular, enzyme-based, microsomal aromatase bioassay
with results from a cell-based aromatase bioassay for active compounds.

| | Noncellular Bioassay | | | Cell-based Bioassay | | | |
|---|---|---|---|---|---|---|---|
| | PCA | | | PCA | | Cytotoxicity | |
| Compound Name | 20 μg/mL | SEM | IC50 (μM) | 50 μM | SEM | % cell surv. | SEM |
| methanol extract | 18.9 | 0.76 | | 24.1 | 3.06 | 65.7 | 1.23 |
| chloroform extract | 29.8 | 1.97 | | 16.5 | 2.39 | 58.5 | 1.18 |
| cudraxanthone G | 57.8 | 0.47 | | | | | |
| 8-deoxygartanin | 82.6 | 0.61 | | | | | |
| garcinone D | 10.0 | 0.87 | 5.2 | 50.7 | 0.78 | 89.4 | 1.32 |
| garcinone E | 23.9 | 0.50 | 25.1 | 32.3 | 3.23 | 52.3 | 0.73 |
| gartanin | 75.9 | 2.84 | | | | | |
| 8-hydroxycudraxanthone G | 55.1 | 0.87 | | | | | |
| 1-isomangostin | 52.6 | 1.06 | | | | | |
| α-mangostin | 22.2 | 0.93 | 20.7 | 59.4 | 3.49 | 18.4 | 0.70 |
| γ-mangostin | 4.7 | 1.20 | 6.9 | −0.5 | 1.45 | 30.5 | 0.54 |
| mangostinone | 78.8 | 1.07 | | | | | |
| smeathxanthone A | 80.8 | 1.01 | | | | | |
| tovophylline A | 74.7 | 1.62 | | | | | |
| DMSO^{a} | 100.0 | 0.44 | | 100.0 | 14.71 | 100.0 | 1.19 |
| AG^{b} | 15.7 | 0.55 | | | | | |
| LET^{c} | | | | 7.4 | 1.05 | | |

^{a}Dimethyl sulfoxide (DMSO), blank/negative control for both noncellular and cell-based bioassay.
^{b}Aminoglutethimide (AG), positive control for noncellular bioassay.
^{c}Letrozole (LET), positive control for cell-based bioassay.

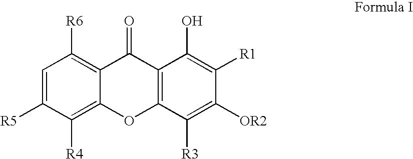

R1=prenyl or a hydrocarbon of five or more carbons or esters thereof
R2= —H, —CH3, —CH₂— or a hydrocarbon or esters thereof
R3= —H, -prenyl or a hydrocarbon of five or more carbons or esters thereof
R4= —H, —OH, —OCH3, a prenyl group or a hydrocarbon of five or more carbons or esters thereof
R5=H, or —OH.
R6 is —H, —OH, —OCH3, a prenyl group, a hydrocarbon of five or more carbons, a hydroxlyated hydrocarbon of 5 carbons or more, or esters thereof;

along with pharmaceutically acceptable salts thereof.

Formula I is exemplary of the molecules identified herein as xanthones. Of the 12 xanthones tested, compounds 3, 4, 8, and 9 demonstrated substantial inhibition of aromatase, and are the only compounds bearing an hydroxy group at C-1, C-3 (R2 in Compound A) and C-6, a prenyl at C-2 (R1 in Compound A) and a five carbon substituent at C-8. Compound 7 is similar, but the prenyl group at C-2 is absent, and instead is cyclized with the hydroxy group at C-1. Compound 7 exhibits aromatase inhibiting activity, but less so that compounds 3, 4, 8, and 9. Compound 5 has even less aromatase inhibiting activity, lacking the hydroxy group at C-6, instead having a hydroxyl group at C-5.

Based on the forgoing, a modified structure that cyclizes a five carbon chain at R5 with the hydroxy group at C-7 would be a promising synthetic compound. Such a ring structure is present in Compound 12, but compound 12 also has a prenyl group at C-5.

For the purposes of this application, the xanthone compounds are numbered as follows:

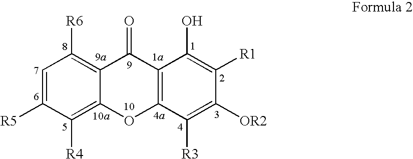

While the xanthones of mangosteen are herein identified to have particular activity in inhibiting the aromatase enzyme, medicinal chemists will recognize the functionalities that are correlated with aromatase inhibiting activity as shown in relation to Compound A.

Of the tested compounds, garcinone D, garcinone E, α-mangostin, and γ-mangostin are recognized have possessing the greatest aromatiase inhibiting activity. Modification of these compounds at the positions shown to be associated with this activity is predicted to yield a library of compounds with varying levels of activities useful for inhibiting aromatase in human patients.

The activity of these extracts and compounds is further demonstrated in a more biologically relevant assay, using breast cancer cells. Active compounds were further screened in a cell-based aromatase bioassay using SK-BR-3 hormone-independent breast cancer cells that overexpress aromatase. Mangosteen extracts and xanthones were found to inhibit aromatase in a dose-dependent manner in SK− BR-3 breast cancer cells. Comparison of the potency of aromatase inhibition in breast cancer cells with cytotoxicity for SK-BR-3 cells resulted in a finding of five-fold more potent aromatase inhibition than cytotoxicity.

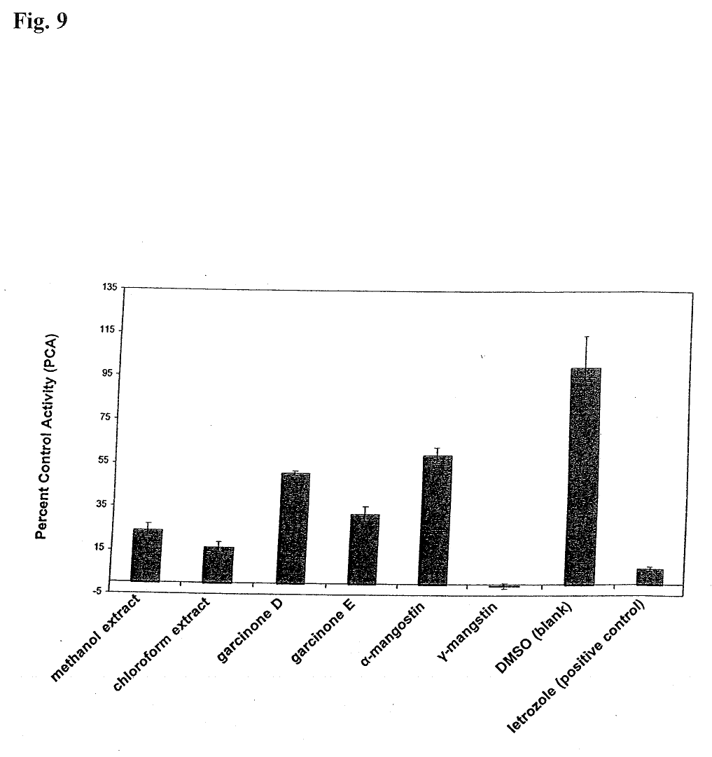

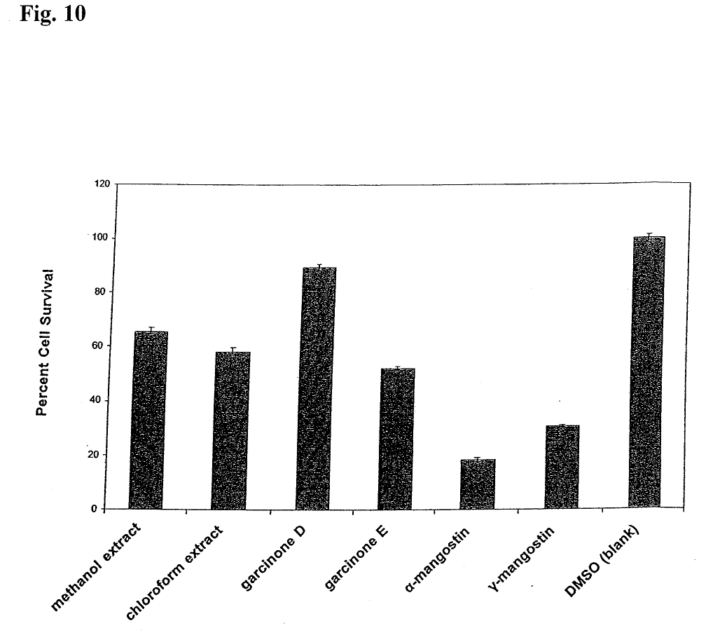

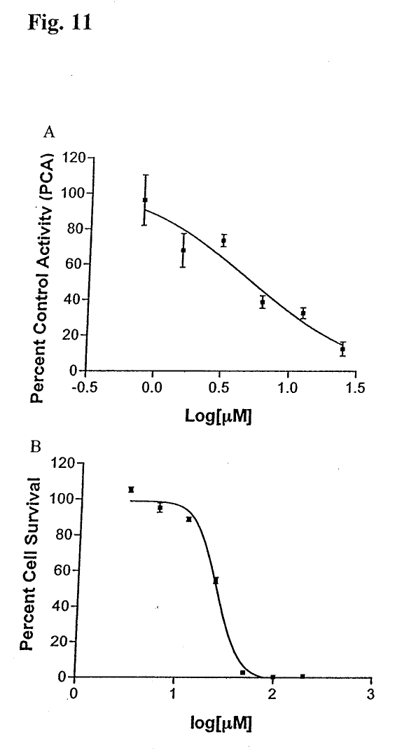

Active compounds were then tested in a secondary cell-based assay, using SK-BR-3 hormone-independent human breast cancer cells that overexpress the aromatase enzyme. FIG. 9 shows percent control activity of various compositions in a SK-BR-3 cell-based aromatase bioassay. In FIG. 9, DMSO=dimethylsulfoxide, blank/negative control; letrozole=positive control, and the other compounds are as identified at 50 μM. γ-Mangostin was found to strongly inhibit aromatase in cells (−0.5 PCA), while garcinone E was found to moderately inhibit aromatase in cells (32.3 PCA). However, γ-mangostin was also found to be fairly cytotoxic in SK-BR-3 cells, complicating the determination if the aromatase inhibition was due to actual activity or the result of low cell survival. FIG. 10 shows the percent cell survival following treatment with compositions from mangosteen in the SK-BR-3 cytotoxicity bioassay. As is apparent, certain of these compositions when delivered at 50 μM display appreciable cytotoxicity. To further understand this effect, γ-mangostin was further subjected to IC50 testing in both the SK-BR-3 cell-based aromatase assay and SK-BR-3 cell-based cytotoxicity assay. FIG. 10 shows IC50 curves for γ-mangostin in (A) SK-BR-3 aromatase bioassay (IC50=4.97 μM), and (B) SK-BR-3 cytotoxicity bioassay (IC50=25.99 μM). As shown in FIG. 11, the IC50 of γ-mangostin in the cell-based AI assay was determined to be 4.97±1.9 μM, while the IC50 in the cell-based cytotoxicity assay was found to be 25.99±1.0 μM.

The concept of a chemopreventive index (CI), provides an idea of the therapeutic efficacy of a composition. The CI is computed using the equation CI=cytotoxicity IC50/aromatase inhibition IC50. This concept is further described by Pezzuto et al., 2005. The CI for γ-mangostin was calculated as 5.2. This CI for γ-mangostin demonstrates that this composition is predicted to be useful as an aromatase inhibitor.

Xanthones produced by chemical synthesis have only recently been tested for their ability to inhibit aromatase (Recanatini et al., 2001; Recanatini et al., 2002; Pinto et al., 2005). Identified synthetic xanthones were active in the nanomolar range, but have not yet undergone extensive evaluation using additional in vitro as well as in vivo and preclinical models. Xanthones most likely inhibit aromatase in a manner similar to the mode of action of nonsteroidal AIs, exhibiting noncompetitive, reversible binding of the aromatase enzyme through interaction with the aromatase heme iron, a typical component of cytochrome P450 dependent enzymes.

As described above, mangosteen is commonly utilized in Southeast Asian traditional medicine for stomach ailments (pain, diarrhea, dysentery, ulcers), as well as to treat infections and wounds, and while known to generally have a variety of beneficial effects, including as an antioxidant, mangosteen is not generally recognized as a dietary supplement useful for preventing or treating neoplasias. Mangosteen products have been attributed to possess such numerous and varied pharmacological effects, such that a specific mode of action, other than providing scavengers for oxygen free radicals and activated metabolites has not been noted. Xanthones as embodied herein acting as inhibitors of the initiation or progression of neoplasias and or as a modulator of aromatase activity are not previously known.

The major isolates from mangosteen, α-mangostin and γ-mangostin, were found to inhibit 7,12-dimethylbenz[α]anthracene-induced (DMBA-induced) preneoplastic lesions in a mouse mammary organ culture (MMOC) assay as described in (Jung et al., 2006). The major isolates from mangosteen, α-mangostin (1.37% w/w yield from mangosteen pericarps) and γ-mangostin (0.26% w/w yield from mangosteen pericarps) were also found to be strong antioxidants using a peroxynitrite scavenging assay. As embodied in the disclosure herein, certain xanthones from mangosteen act as potent aromatase inhibitors in both noncellular and cell-based AI assays.

While not previously recognized, the relatively high concentration of xanthones in mangosteen botanical dietary supplements may be sufficient to provide a moderate amount of aromatase inhibitors, and may thus be useful for hormone-dependent breast cancer chemoprevention in postmenopausal women. Consumption of moderate amounts of botanical dietary supplements from mangosteen may supply minimal amounts of xanthone aromatase inhibitors that provide a chemopreventive benefit to those at risk of estrogen dependent cancers. A continuing problem with supplying chemotherapeutic agents from natural sources is that there is great difficulty in assuring that a botanically derived supplement is providing a composition that best presents the beneficial agents. Identification of an active compound thus provides a method of standardizing a botanically derived supplement for at least one identifiable biologically active compound, providing reassurance that the supplement has potential efficacy for an identified benefit. Thus, mangosteen supplements could be standardized to provide a given amount of one or more xanthone derivatives. For instance, a mangosteen supplement could be standardized to contain a given and or minimum quantity per dose of γ-mangostin, and or garcinone E, and or one of the other compounds identified in FIGS. 4, 5, and 12.

Xanthones isolated from mangosteen, by acting as potent aromatase inhibitors as disclosed herein, are expected to provide an advantageous source of aromatase inhibitors for breast cancer chemoprevention and chemotherapy, along with for similar effects on other estrogen dependent cancers and disease. As such, aromatase inhibitors (AIs) can be utilized as either anticancer agents or for cancer chemoprevention. In particular, those women who are genetically predisposed to be at high risk for developing breast cancer may benefit from utilization of aromatase inhibitors. However, the use of AIs for cancer chemotherapy or chemoprevention is limited to postmenopausal women or premenopausal women who have undergone ovarian ablation.

As another example of the useful compounds that can be identified using the assays described herein, several compounds were isolated and characterized from from Licorice (Glycyrrhiza glabra L.). Licorice has a long history of use as a food and a food flavoring. There is broad interest in understanding the composition of botanical products such as licorice, for example, and to understand bioactive compounds that may be present in such products which may be useful for chemopreventive or chemotherapeutic uses.

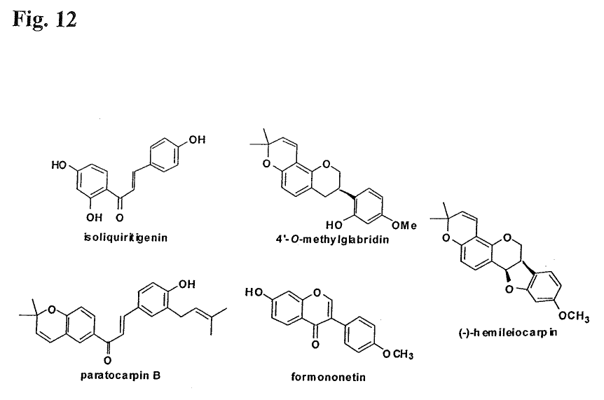

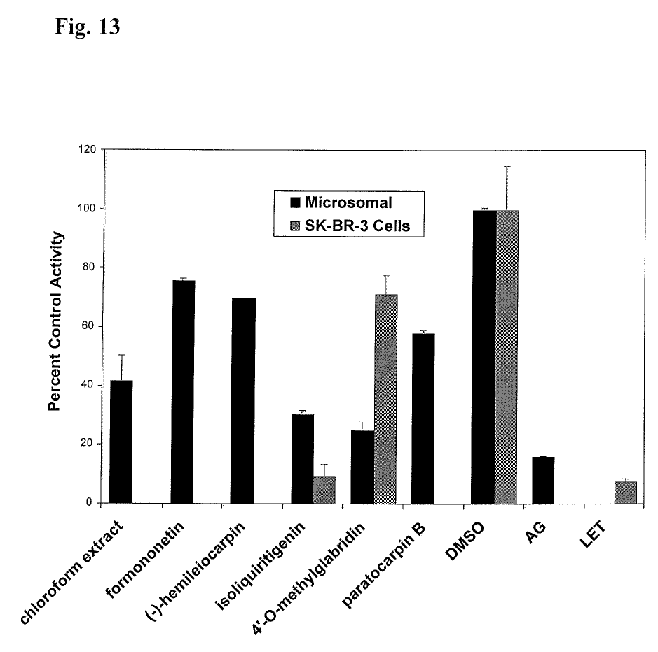

FIG. 12 shows the structures of various compounds tested from Licorice. These include isoliquiritigenin, 4′O-methylglabridin, (−)-hemileiocarpin, paratocarpin B, and formonotetin. These compounds were analyzed using the microsomal assay and by their activity when used with SK-BR-3 cells, as described above and in the Examples that follow. FIG. 13 shows a plot of the results from an aromatase bioassay Licorice extracts and compounds, including those compounds shown in FIG. 12. As shown in FIG. 13, the compounds isoliquiritigenin, 4′O-methylglabridin, paratocarpin B, in particular, show results that are supportive of these compounds having utility as aromatase inhibitors.

Formulations and Methods of Administration

The extracts disclosed and compositions derived therefrom can be administered to a human subject in any suitable form. For example, the extracts and compositions are sufficiently stable such that they can be readily prepared in a form suitable for adding to various foodstuffs including, for example, juice, fruit drinks, carbonated beverages, milk, nutritional drinks (e.g., Ensure™, Metracal™), ice cream, breakfast cereals, biscuits, cakes, muffins, cookies, toppings, bread, bagels, fiber bars, soups, crackers, baby formulae (e.g., Similac™), teas, salad dressings, cooking oils, and meat extenders.

In addition, extracts and compositions derived therefrom can be formulated as a pharmaceutical composition (e.g., a medicinal drug) for the treatment of specific disorders. In one embodiment, mangosteen extracts, synthetic analogs and compositions derived therefrom can be formulated as a dietary supplement. Suitable additives, carriers and methods for preparing such formulations are well known in the art.

One advantage of utilizing extracts or specific compounds described herein over simply consuming mangosteen fruit juice is a reduction in the quantity of free sugars that are present in juice. In particular, free sugars such as fructose and sucrose are present in relatively high concentrations. By extracting only those most beneficial components of the mangosteen plant, and providing that composition to patients, most of the additional sugars and calories are removed, while making consumption of a therapeutically effective amount practicable.

Pharmaceutical compositions may take the form of tablets, capsules, emulsions, suspensions and powders for oral administration, sterile solutions or emulsions for parenteral administration, sterile solutions for intravenous administration and gels, lotions and cremes for topical application, and suppositories for colorectal or cervical administration. The pharmaceutical compositions may be administered to humans and animals in a safe and pharmaceutically effective amount to elicit any of the desired results indicated for the compounds and mixtures described herein.

The pharmaceutical compositions of this invention typically comprise a pharmaceutically effective amount of a mangosteen extract, a mangosteen fruit extract or fraction thereof, or an analog or synthetic analog thereof, containing, for example, an extract or compounds with anti-aromatase activity, and, if suitable, a pharmaceutically acceptable carrier. Such carriers may be solid or liquid, such as, for example, cornstarch, lactose, sucrose, olive oil, or sesame oil. If a solid carrier is used, the dosage forms may be tablets, capsules or lozenges. Liquid dosage forms include soft gelatin capsules, syrup or liquid suspension.

Therapeutic and prophylactic methods comprise the steps of treating patients or animals in a pharmaceutically acceptable manner with the compositions and mixtures described herein.

The pharmaceutical compositions of this invention may be employed in a conventional manner for the treatment and prevention of any of the aforementioned diseases and conditions. Such methods of treatment and prophylaxis are well-recognized in the art and may be chosen by those of ordinary skill in the art from the available methods and techniques. However, lower or higher dosages may be employed. The specific dosage and treatment regimens selected will depend upon factors such as the patient's or animal's health, and the severity and course of the patient's (or animal's) condition and the judgment of the treating physician. In another preferred embodiment, the xanthones disclosed herein are delivered at 25 mg/day, 50 mg/day, or 100 mg/day.

The mangosteen extracts compositions derived therefrom also can be used in combination with conventional therapeutics used in the treatment or prophylaxis of any of the aforementioned diseases. Such combination therapies advantageously utilize lower dosages of those conventional therapeutics, thus avoiding possible toxicity incurred when those agents are used alone. For example, other nutrients or medications, for example, estrogen lowering drugs, chemotherapeutic agents, and/or radiotherapy.

The disclosure may be better understood by reference to the following examples, which are by no means to be construed as limiting.

### EXAMPLES

### Example 1

### Definitions

The term “analog” as in “a compound or synthetic analog thereof”, is intended to include compounds that are structurally similar but not identical to the compound, but retain some or all of the beneficial properties of the compound.

As used herein the term “anti-cancer activity” or “anti-cancer properties” refers to the inhibition (in part or in whole) or prevention of a cancer as defined herein. Anti-cancer activity includes, e.g., the ability to reduce, prevent, or repair genetic damage, modulate undesired cell proliferation, modulate misregulated cell death, or modulate mechanisms of metastasis (e.g., ability to migrate).

The term “antioxidants” includes chemical compounds that can absorb an oxygen radical, e.g., ascorbic acid and phenolic compounds.

The term fruit extract refers to fruits which have been transformed in some manner, for example, pureed, freeze-dried and particularly by modifications resulting from freezing and dehydration resulting in a freeze-dried extract enriched for antioxidant activity and other beneficial compounds. In general a fruit extract is defined to include a mixture of a wide variety of compounds from the originating fruit.

The term “fraction” refers to a composition that has been separated into pools of substituent components of the fractionated composition, with such fractionation being performed by a variety of means, including, but not limited to density, solubility, mobility and chromatographic methods. Further separation of a fraction by alternative means of fractionation may yield subfractions and compounds.

The term “cancer” or “malignancy” are used interchangeably and include any neoplasm (e.g., benign or malignant), such as, for instance, a carcinoma (i.e., usually derived from epithelial cells, e.g., skin cancer,) or sarcoma (usually derived from connective tissue cells, e.g., a bone or muscle cancer) or a cancer of the blood, such as a erythroleukemia (a red blood cell cancer) or leukemia (a white blood cell cancer). A “malignant” cancer (i.e., a malignancy) can also be metastatic, i.e., have acquired the ability to transfer from one organ or tissue to another not directly connected, e.g., through the blood stream or lymphatic system.

The term “dietary supplement” includes a compound or composition used to supplement the diet of an animal or human.

The term “foodstuff” includes any edible substance that can be used as or in food for an animal or human. Foodstuffs also include substances that may be used in the preparation of foods such as cooking oils or food additives. Foodstuffs also include dietary supplements designed to, e.g., supplement the diet of an animal or human.

The terms “health promoting”, “therapeutic” and “therapeutically effective” are used interchangeably herein, and refer to the prevention or treatment of a disease or condition in a human or other animal, or to the maintenance of good health in a human or other animal, resulting from the administration of a berry extract (or fraction thereof) of the invention, or a composition derived therefrom. Such health benefits can include, for example, nutritional, physiological, mental, and neurological health benefits.

The term “isolated” refers to the removal or change of a composition or compound from its natural context, e.g., the mangosteen plant.

The term “pharmaceutical composition” or “therapeutic composition” refers to a composition formulated for therapeutic use and may further comprise, e.g., a pharmaceutically acceptable carrier. The term “pharmaceutically effective amount” refers to an amount effective to achieve a desired therapeutic effect, such as lowering tumor incidence, metastasis, immunoregulatory diseases, cancer, or signs of aging.

The phrase “prevention of disease” relates to the use of the invention to reduce the frequency, severity, or duration (of disease) or as a prophylactic measure to reduce the onset or incidence of disease.

### Example 2

### General Experimental Procedures

Methanol and chloroform-soluble extracts of Garcinia mangostana L. (Clusiaceae) (mangosteen) were prepared and individual xanthones were isolated as described in a Jung et al., 2006.

Melting points were determined on a Thomas-Hoover capillary melting point apparatus and are uncorrected. The UV spectra were obtained with a Beckman DU-7 spectrometer, and the IR spectra were run on an ATI Mattson Genesis Series FT-IR spectrophotometer. NMR spectroscopic data were recorded at room temperature on a Bruker Advance DPX-300 or a DRX-400 MHz spectrometer with tetramethylsilane (TMS) as internal standard. Standard pulse sequences were employed for the measurement of 2D NMR spectra (1H-1H COSY, HMQC, HMBC, and NOESY). Electrospray ionization (ESI) mass spectrometric analysis was performed with a 3-T Finnigan FTMS-2000 Fourier transform mass spectrometer. Column chromatography was carried out with Purasil (230-400 mesh, Whatman, Clifton, N. J.). Analytical thin-layer chromatography (TLC) was performed on 250 μm thickness Merck Si gel 60 F254 aluminum plates. A SunFire PrepC180BD column (5 μm, 150×19 mm i.d., Waters, Milford, Mass.) and a SunFire PrepC18 guard column (5 μm, 10×19 mm i.d., Waters) were used for HPLC, along with two Waters 515 HPLC pumps and a Waters 2487 dual λ absorbance detector.

Chemicals. L-Ascorbic acid, DL-2-amino-3-mercapto-3-methyl-butanoic acid (DL-penicillamine), diethylenetriaminepentaacetic acid (DTPA), and 3-morpholinosydnonimine (SIN-1) were purchased from Sigma Chemical Co. (St. Louis, Mo.). Dihydrorhodamine 123 (DHR 123) and peroxynitrite (ONOO—) sodium salt were obtained from Molecular Probes (Eugene, Oreg.) and Cayman Chemicals Co. (Ann Arbor, Mich.), respectively. Radiolabeled [1β-³H]androst-4-ene-3,17-dione was purchased from NEN Life Science Products (Boston, Mass.). Radioactivity was counted on a LS6800 liquid scintillation counter (Beckman, Palo Alto, Calif.). Scintillation cocktail 3a70B was purchased from Research Prospect International Corporation (Mount Prospect, Ill.). SK-BR-3 hormone-independent human breast cancer cells were obtained from American Type Culture Collection (Rockville, Md.). All other chemicals and reagents were purchased from Sigma-Aldrich (St. Louis, Mo.).

Plant Material. The freeze-dried powder of the pericarp of G. mangostana used in this study was obtained from Nature's Sunshine Products, Inc. A representative sample (lot 0112824) was deposited as a powder in the Division of Medicinal Chemistry and Pharmacognosy, College of Pharmacy, The Ohio State University.

Extraction and Isolation. The dried and milled pericarp of G. mangostana (1 kg) was extracted by maceration with MeOH (3×5 L) at room temperature, for 3 days each. After filtration and evaporation of the solvent under reduced pressure, the combined crude methanolic extract (324.3 g) was suspended in H2O (700 mL) to produce an aqueous solution, then partitioned in turn with n-hexane (3×500 mL), CH₂Cl₂ (3×500 mL), EtOAc (3×500 mL), and n-BuOH (3×500 mL) to afford dried n-hexane (36.9 g), CH₂Cl₂ (111.2 g), EtOAc (69.3 g), n-BuOH (141.7 g), and H2O-soluble (˜7.3 g) extracts. The CH2—Cl2-soluble partition was found to have significant antioxidant activity in a ONOO— scavenging bioassay. Therefore, this extract was selected for further detailed purification.

### Example 3

### Characterization of Samples

Compounds were isolated from the pericarp of G. mangostana, were evaluated individually in a QR induction assay. The structures of the compounds were identified by physical and spectroscopic data measurement ([α]_{D}²³, ¹H NMR, ¹³C NMR, DEPT, 2D NMR, and MS) and by comparing the data obtained with those of published values, as 1,3,7-trihydroxy-2,8-di-(3-methylbut-2-enyl)xanthone (Mahabusarakam et al., 1987), mangostanin (Nilar and Harrison, 2002), and α-mangostin (Sen et al., 1982).

Compound 21 [1,2-dihydro-1,8,10-trihydroxy-2-(2-hydroxypropan-2-yl)-9-(3-methylbut-2-enyl)furo[3,2-a]xanthen-11-one, with the configurations of C-1″ and C-2″ unresolved], was obtained as yellow powder, and the elemental composition was inferred from a sodiated ion peak at m/z 435.1425 (calcd for C₂₃H₂₄O₇Na, 435.1420) in the HRESI-TOF MS. The ¹H NMR spectrum of 21 exhibited ortho-coupled signal resonances at δ_{H} 7.36 (1H, d, J=8.9 Hz, H-6), and 7.50 (1H, d, J=8.9 Hz, H-5), a singlet signal at δ_{H} 6.45 (1H, s, H-4), and two aromatic hydroxyl peaks at δ_{H} 13.08 (OH-1) and 11.10 (OH-3), assignable to a xanthone moiety (Hano et al., 1990). A 3-methylbut-2-enyl group was also observed at δ_{H} 1.72 (3H, s, H-4′), 1.62 (3H, s, H-5′), 3.23 (1H, d, J=6.8 Hz, H-1′), and 5.18 (1H, brt, J=6.0 Hz, H-2′), as well as a 2-(1-hydroxy-1-methylethyl)-2,3-dihydrofuran-3-ol moiety from resonances at δ_{H} 5.80 (1H, brt, J=3.7 Hz, H-1″), 5.14 (1H, d, J=4.0 Hz, OH-1″), 4.70 (1H, s, OH-3″), 4.32 (1H, d, J=3.6 Hz, H-2″), 1.18 (3H, s, H-4″) and 1.09 (3H, s, H-5″). The HMBC correlation of the signal δ_{H} 3.23 (H-1″) to δ_{C} 159.5 (C-1), 109.9 (C-2), and 163.6 (C-3), as well as those at δ_{H} 6.45 (H-4) to δ_{C} 163.5 (C-3), 102.4 (C-1a), and 155.2 (C-4-a), were suggestive of the connectivity of a 3-methylbut-2-enyl side chain at C-2. A 2-(1-hydroxy-1-methylethyl)-2,3-dihydrofuran-3-ol group was positioned between C-7 and C-8 by the observed two or three-bond correlations from signals at δ_{H} 7.36 (H-6) to δ_{C} 156.6 (C-7), 126.4 (C-8), and 150.2 (C-10a), δ_{H} 7.50 (H-5) to 156.6 (C-7), 117.2 (C-9a), and 150.2 (C-10a), and δ_{H} 4.32 (H-2″) to δ_{C} 156.6 (C-7), and 20.9 (C-1″). Thus, the structure of this compound was elucidated as 1,2-dihydro-1,8,10-trihydroxy-2-(2-hydroxypropan-2-yl)-9-(3-methyl but-2-enyl)furo[3,2-a]xanthen-11-one, with the configurations of C-1″ and C-2″ unresolved.

The molecular formula of a second compound, 22, was assigned as C₂₃H₂₄O₆, from the observed sodiated ion at m/z 419.1475 (calcd for C₂₃H₂₄O₆Na, 419.1471) in the HRESI-TOF MS. The ¹H NMR spectroscopic data showed the presence of a penta-substituted xanthone moiety [δ_{H} 7.30 (2H, s, H-5 and H-6), and 6.40 (1H, s, H-4)], a 2-(1-hydroxy-1-methylethyl)-2,3-dihydrofuran ring [δ_{H} 4.75 (1H, t, J=8.5 Hz, H-2′), 3.06 (2H, d, J=8.5 Hz, H-1′), 1.32 (3H, s, H-4′) and 1.15 (3H, s, H-5′)], and a 3-methylbut-2-enyl group [δ_{H} 5.18 (1H, brt, J=5.9 Hz, H-2″, 4.02 (2H, d, J=6.0 Hz, H-1″), 1.76 (3H, s, H-4″), and 1.60 (3H, s, H-5″)], which were similar to those of mangostanin except for the absence of a hydroxyl group at C-6 and the occurrence of a methoxy group at C-7. Furthermore, HMBC correlations were used to confirm this structure. Thus, the long-range connections of δ_{H} 4.02 (H-1″) to δ_{C} 127.0 (C-8), 123.4 (C-2″), 151.5 (C-7), 117.9 (C-9a), and 130.3 (C-3″), and δ_{H} 7.30 to δ_{C} 127.0 (C-8) and 117.9 (C-9a) suggested that a 3-methylbut-2-enyl group was located at C-8. The presence of a 2-(1-hydroxy-1-methylethyl)-2,3-dihydrofuran unit was proposed by the proton to carbon connectivities of δ_{H} 3.06 (H-1′) to δ_{C} 157.0 (C-1), 107.7 (C-2), and 167.2 (C-3), as well as δ_{H} 6.40 (H-4) to 167.2 (C-3), 107.7 (C-2), 156.8 (C-4a), and 103.4 (C-1a). Therefore, the structure of compound 22 was determined as 2,3-dihydro-4,7-dihydroxy-2-(2-hydroxypropan-2-yl)-6-(3-methyl but-2-enyl)furo[3,2-b]xanthen-5-one (6-deoxy-7-de-methylmangostanin).

The isolated compounds described above, together with cudraxanthone G, 8-deoxygartanin, garcinone D, garcinone E, gartanin, 8-hydroxycudraxanthone G, 1-isomangostin, γ-mangostin, mangostinone, tovophyllin A, and smeathxanthone A, were tested in an in vitro screening assay using murine hepatoma cells (Hepa 1c1c7) for the induction of quinone reductase (QR). Of all tested compounds, only compounds 21-24 were found to induce QR activity as shown in Table 3. The CD (concentration required to double QR induction activity) values of compounds 21-24 (1.3, 2.2, 0.68, and 0.95 μg/mL, respectively) were comparable to that of isoliquiritigenin (1.1 μg/mL), used as a positive control. Moreover, compound 21 exhibited a larger chemoprevention index (CI=IC50/CD) than isoliquiritigenin, which has shown evidence of cancer chemopreventive effects in in vivo models (Baba et al., 2002; Chin et al., 2007). Thus compound 21 is a candidate for use as a cancer chemopreventive agent.

Additionally, the antioxidant capacity of these xanthones was evaluated in a hydroxyl-radical scavenging assay. Only γ-mangostin (26) from the library of xanthones available was found to be active (IC50, 0.20 μg/mL) whereas all other compounds were inactive (IC50>10 μg/mL), including the QR-inducing agents 21-24. The antioxidant potency of 26 in the hydroxyl-radical scavenging assay used is comparable to those of the positive controls used, gallic acid (IC50, 1.0 μg/mL), quercetin (IC50, 0.38 μg/mL), and vitamin C (IC50, 0.40 μg/mL), as well as data obtained in a recently published study on this same xanthone (Yu et al., 2007).

### Example 4

### Molecular Characterization of Mangosteen Xanthones

A molecular formula of C24H2606 was determined for compound 1 by its HRESIMS (m/z 433.16114 [M+Na]+). The ¹H NMR spectrum revealed two downfield singlets at δH 11.22 and 12.16, suggesting the presence of two hydrogen-bonded hydroxy groups in the molecule of 1. The 1H NMR spectrum of this compound also displayed the characteristic signals of two ortho-coupled aromatic protons at δH 7.25 (1H, d, J) 9.0 Hz, H-6) and 6.69 (1H, d, J) 9.0 Hz, H-7), two olefinic protons at δH 5.24 (2H, m, H-2′ and H-2″), one methoxy group at δH 3.81 (3H, OMe-3), and four tertiary methyls at δH 1.87 (3H, s, H-5″), 1.81 (3H, s, H-5′), 1.74 (3H, s, H-4″), and 1.71 (3H, s, H-4′). The 13C NMR spectrum of compound 1 showed 24 resonance signals. The presence of two 3-methylbut-2-enyl functionalities in compound 1 could be assigned by interpretation of its 1H and 13C NMR spectroscopic data as well as the correlations observed in the 1H-1H COSY, HMQC, and HMBC spectra. In addition to the signals of these two prenyl groups and the signal of a typical methoxy substituent group, only 11 carbon resonance signals composed of two aromatic rings and one doubly conjugated carbonyl carbon (δC 185.4) remained for compound 1. These NMR data suggested that compound 1 is a prenylated xanthone derivative. The two downfield hydrogen-bonded hydroxy singlets at δH 11.22 and 12.16 suggested the locations of two of the three hydroxy groups to be at C-1 and C-8 in the molecule of 1. In the HMBC spectrum, correlations were observed from H-6 to C-5, C-8, and C-10a, from H-7 to C-5, C-6, C-8, and C-9a, from OMe-3 to C-3, and from both H-1′ and H-1″ to C-3. These correlations were used to assign the positions of two prenyl units and the methoxy group. Therefore, compound 1 was determined to be 8-hydroxycudraxanthone G.

A sodiated molecular ion peak at m/z 447.14323 [M+Na]+ in its HRESIMS was used to assign a molecular formula of C24H2407 for compound 2. The UV (λmax at 243, 320, and 354 nm) and IR [vmax at 3365 (OsH), 1608 (CdO), and 1578 (aromatic ring) cm-1] spectroscopic data of compound 2 were very similar to those of 1. The 1H and 13C NMR spectroscopic data suggested that compound 2 is also a prenylated xanthone. In the 1H NMR spectrum of 2, only one downfield singlet for a hydrogen-bonded hydroxy group was displayed at δH 13.50 (OH-1). In addition to a methoxy group and the signals of the xanthone skeleton, ten other resonances were shown in the 13C NMR spectrum of 2. By interpretation of the chemical shifts and splitting patterns as well as the observed 2D NMR (1H-1 H COSY, HMQC, and HMBC) correlations of the nonskeletal protons and carbons, the two prenyl units in the molecule of 2 were determined as 3-methylbut-2-enyl and 2-oxo-3-methylbut-3-enyl, respectively. On the basis of the above-mentioned NMR data analysis and the determined molecular formula, the presence of three hydroxyl groups could be deduced. The positions of all substituents, namely, one methoxy group, two prenyl units, and three hydroxy groups, were assigned by careful analysis of the correlations obtained in the HMBC spectrum. The observed key HMBC correlations for the structure assignment were from OH-1 to C-1a, C-1, and C-2, from H-1′ to C-1, C-2, and C-3, from H-1″ to C-7, C-8, and C-9a, and from the methoxy singlet at δH 3.73 to C-7. Hence, compound 2, mangostingone, was determined to be 1,3,6-trihydroxy-7-methoxy-2-(3-methylbut-2-enyl)-8-(2-oxo-3-methylbut-3-enyl)-xanthone.

### Example 5

### Purification of Extracts

The CH₂Cl₂-soluble extract was subjected to chromatography over a silica gel column, eluted with CHCl3/MeOH (from 100:1 to 1:1), to give 21 fractions (FO01-21). F08 (200 mg) was chromatographed over a silica gel column with a n-hexane/EtOAc solvent system (20:1 to pure EtOAc) to give ten subfractions (F0801-F0810). Tovophyllin A (14; 10 mg) was obtained as a yellow solid from the solution (CHCl3/MeOH, ˜10:1) of F0807. Subfractions F0804-F0806 were combined and successively chromatographed over a reversed-phase HPLC column with H2O/CH3CN (15:85) at a flow rate of 7.0 mL/min to afford cudraxanthone G (3; 5 mg; tR) 34.0 min) and 8-hydroxycuderaxan-thone G (1; 6 mg; tR) 42.5 min). A portion of fraction F10 (600 mg of 3.4 g) was chromatographed over a silica gel column with a n-hexane/EtOAc solvent system (20:1 to pure EtOAc) to yield the pure compounds 8-deoxygartanin (4; 30 mg) and gartanin (8; 340 mg).

Garcinone E (7; 30 mg) was isolated from F11 by silica gel column chromatography with n-hexane/CH₂Cl₂/EtOAc (65:30:5) as the eluting solvent mixture. R-Mangostin (10; 13 g) was isolated as a major component from combined fractions F12 (4.8 g) and F13 (20 g) by silica gel chromatography eluted with n-hexane/EtOAc (6:1) and on Sephadex LH-20 column chromatography with pure MeOH as solvent. The subfractions of F13 were then combined and chromatographed over a silica gel column eluted with n-hexane/EtOAc (5:1 to EtOAc) to give an additional amount of R-mangostin (10, 650 mg) and the further subfractions, F1301-F1305. Subfraction F1303 was finally purified by semipreparative reversed-phase HPLC [H2O/CH3CN (30:70); flow rate) 6.0 mL/min] to afford a minor new compound, mangostingone (2; 1.2 mg; tR) 15.8 min). Fraction F17 (3.8 g) was chromatographed over a Sephadex LH-20 column using MeOH as eluent, yielding seven subfractions (F1701-F1707).

F1702 (200 mg) was purified over a silica gel column with n-hexane/EtOAc (4:1) as solvent system to afford 1-isomangostin (9, 35 mg) and garcimangosone B (5, 3 mg), in order of polarity. F1705 was separated using a semipreparative reversed-phase HPLC column with H2O/CH3CN (15:85) at a flow rate of 7.0 mL/min to give mangostinone (12; 6 mg; tR) 28.0 min) and smeathxanthone A (13; 8 mg; tR) 45.0 min). F1706 was purified with a Sephadex LH-20 column using pure MeOH as solvent, to give γ-mangostin (11, 600 mg). Fraction F18 was fractionated over a silica gel column with CHCl3/acetone (40:1) as solvents, resulting in 12 subfractions (F1801-F1812). The major subfraction, F1805 (6 g), was chromatographed over a Sephadex LH-20 column, eluting with pure MeOH, to afford another major isolate, γ-mangostin (11; 2 g), and seven subfractions (F180501-F180507). F180502 (100 mg) was purified over a silica gel column with CHCl3/acetone (35:1) as solvent, to afford an additional amount of 1-isomangostin (9; 20 mg). F180504 (90 mg) was chromatographed over a reversed-phase silica gel column eluted with MeOH/H2O (7:3), to yield garcinone D (6; 10 mg).

8-Hydroxycudraxanthone G (1) was obtained as a yellow solid: UV (MeOH) Amax (log □) 238 (4.28), 263 (4.38), 279 (4.34), 351 (3.97) nm; IR (dried film) vmax 3384, 1623, 1584, 1490, 1217, 1098 cm-1; 1H NMR (300 MHz, CDCl3) δ 12.16 (OH), 11.22 (OH), 7.25 (1H, d, J) 9.0 Hz, H-6), 6.69 (1H, d, J) 9.0 Hz, H-7), 5.24 (2H, m, H-2′ and H-2″), 3.81 (3H, s, OCH3-3), 3.54 (2H, d, J) 6.2 Hz, H-1″), 3.41 (2H, d, J) 6.9 Hz, H-1′), 1.87 (3H, s, H-5″), 1.81 (3H, s, H-5′), 1.74 (3H, s, H-4″), 1.71 (3H, s, H-4′); 13C NMR (75 MHz, CDCl3) δ 185.4 (C-9), 164.4 (C-3), 158.8 (C-1), 153.8 (C-8), 152.7 (C-4a), 142.8 (C-10a), 135.9 (C-5), 132.3 (C-3′), 132.2 (C-3″), 123.0 (C-6), 123.0 (C-2″), 122.2 (C-2′), 118.2 (C-2), 113.0 (C-4), 109.8 (C-7), 107.3 (C-9a), 104.9 (C-1a), 62.1 (OCH3-3), 25.7 (C-4′), 25.5 (C-4″), 23.0 (C-1″), 22.5 (C-1′), 18.0 and 17.9 (C-5′ and C-5″); HRESIMS m/z 433.16114 [M+Na]+(calcd for C24H2606Na+, 433.16216).

Mangostingone (2) was obtained as a yellow solid: UV (MeOH) λmax (log □) 243 (3.84), 320 (3.65), 354 (3.32) nm; IR (dried film) vmax 3365, 1608, 1578, 1465, 1284, 1162, 1081 cm-1; 1H NMR (300 MHz, acetone-d6) δ 13.50 (OH), 6.86 (1H, s, H-5), 6.39 (1H, s, H-4), 6.23 (1H, s, H-4″a), 5.86 (1H, s, H-4″b), 5.24 (1H, t, J) 6.8 Hz, H-2′), 4.75 (2H, s, H-1″), 3.73 (3H, s, OCH3-3), 3.30 (2H, d, J) 6.8 Hz, H-1′), 1.92 (3H, s, H-5″), 1.75 (3H, s, H-4′), 1.61 (3H, s, H-5′); 13C NMR (75 MHz, acetone-d6) δ 199.1 (C-2″), 182.2 (C-9), 163.3 (C-3), 161.4 (C-1), 161.1 (C-6), 156.1 (C-4a), 155.8 (C-10a), 145.8 (C-3″), 145.7 (C-7), 131.4 (C-8), 131.2 (C-3′), 123.6 (C-2′), 123.6 (C-4″), 111.0 (C-9a), 111.0 (C-2), 103.3 (C-5), 103.2 (C-1a), 93.4 (C-4), 61.3 (OCH3-3), 37.9 (C-1″), 25.9 (C-4′), 22.0 (C-1′), 18.1 (C-5″), 17.9 (C-5′); HRESIMS m/z 447.14323 [M+Na]+(calcd for C24H2407Na+, 447.14142).

### Example 6

### Measurement of Peroxynitrite Scavenging Activity

ONOO-scavenging activity was measured by monitoring the oxidation of nonfluorescent DHR 123 to highly fluorescent rhodamine 123 using the modified method of Kooy et al. Briefly, DHR 123 (5 mM) in EtOH, purged with nitrogen, was stored at −80° C. as a stock solution. This solution was not exposed to light, prior to the study. The rhodamine buffer (pH 7.4) consisted of 50 mM sodium phosphate dibasic, 50 mM sodium phosphate monobasic, 90 mM sodium chloride, 5 mM potassium chloride, and 100 μM DTPA. The final concentration of DHR 123 was 5 μM. The buffer in this assay was prepared before use and placed on ice. The concentrations of compounds tested were in the range from 0.2 to 100 μM in 10% DMSO. The background and final fluorescent intensities were measured 5 min after treatment with and without the addition of authentic ONOO— in 0.3 N sodium hydroxide (10 μM) or SIN-1 in deionized water (10 μM). DHR 123 was oxidized rapidly by ONOO—, superoxide anion (O2.-), and nitric oxide (NO.). The fluorescence intensity of oxidized DHR 123 was measured with an LS55 luminescence spectrometer (Perkin-Elmer, Boston, Mass.) at the excitation and emission wavelengths of 480 and 530 nm, respectively. Values of ONOO— scavenging activity (50% inhibition, IC50) were expressed as the mean (n)3) for the final fluorescence intensity minus background fluorescence by the detection of oxidation of DHR 123. DL-Penicillamine was used as a positive control.

### Example 7

### Mouse Mammary Organ Culture Assay

This assay was carried out according to an established protocol disclosed in the art. In brief, 4-week-old BALB/c female mice (Charles River, Wilmington, Mass.) were pretreated for 9 days with 1 μg of estradiol and 1 mg of progesterone. On the 10th day, the mice were sacrificed and the second pair of thoracic mammary glands was dissected on silk and transferred to 60 mm culture dishes containing 5 mL of Waymouth's 752/1 MB medium supplemented with streptomycin, penicillin, and L-glutamine. The glands were incubated for 10 days (37° C., 95% O2 and 5% CO2) in the presence of growth-promoting hormones (5 μg of insulin, 5 μg of prolactin, 1 μg of aldosterone, and 1 μg of hydrocortisone per milliliter of medium). Glands were exposed to 2 μg/mL 7,12-dimethylbenz[a]anthracene (DMBA) between 72 and 96 h. After their exposure, glands were rinsed and transferred to new dishes with fresh medium. The fully differentiated glands were then permitted to regress by withdrawing all hormones except insulin for 14 additional days. Test compounds were present in the medium during days 1-10 of culture; mammary glands were scored for the incidence of lesions.

### Example 8

### Noncellular, Enzyme-Based Aromatase Bioassay

Human placental microsomes were obtained from human term placentas that were processed at 4° C. immediately after delivery from the Ohio State University Medical Center. After washing the placenta with normal saline, connective and vascular tissues were removed. Microsomes were obtained from the remaining tissue as described (Kellis and Vickery, 1987). Aliquots of microsomes were stored at −80° C. until required.

Extracts and compounds were originally screened at 20 μg/mL in DMSO using a noncellular microsomal radiometric aromatase assay, performed as in (O'Reilly et al., 1995). Compounds with poor solubility in DMSO were sonicated and/or heated as needed to improve solubility. Samples [extracts or compounds, DMSO as negative control, or 50 μM (±)-aminoglutethimide (AG) as positive control] were tested in triplicate. Samples were added to 100 nM [1β-³H]androst-4-ene-3,17-dione (400,000-450,000 dpm), 0.1 M potassium phosphate buffer (pH 7.0), 5% propylene glycol, and an NADPH-regenerating system (containing 2.85 mM glucose-6-phosphate, 1.8 mM NADP⁺, and 1.5 units glucose-6-phosphate dehydrogenase). The reactions were initiated by adding 50 μg microsomal aromatase, incubated in a shaking water bath at 37° C., and quenched after 15 minutes using 2 mL CHCl₃. Tubes were vortexed and then centrifuged for 5 minutes. The aqueous layer was removed from each tube and extracted two more times with CHCl₃ to afford an exhaustive extraction. An aliquot of the aqueous layer was then added to 3a70B scintillation cocktail for quantitation of the formation of ³H₂O. Background values were determined using boiled, inactivated microsomal aromatase. Results are given as percent control activity (PCA) calculated using the formula:

PCA=(Sample dpm-DMSO dpm)/(DMSO dpm-Boil dpm)*100 where dpm is disintegrations per min and Boil is the background determined by inactivating the microsomal aromatase by boiling. IC50 values were determined for the active compounds (defined here as <50 PCA) by nonlinear regression using six inhibitor concentrations ranging from 1 μM to 100 μM. IC50 dose-response curves were analyzed using Graphpad Prism (Version 3.0).

### Example 9

### Cell-Based Aromatase Bioassay

Certain extracts and compounds found to be active using the noncellular, enzyme-based radiometric aromatase inhibition assay were further tested at various concentrations in SK-BR-3 hormone-independent human breast cancer cells that overexpress aromatase, using previously described methodology (Natarajan et al., 1994; Richards and Brueggemeier, 2003). SK-BR-3 cell cultures were maintained in custom phenol red-free media containing MEM, Earle's salts, 1.5× amino acids, 2× nonessential amino acids, L-glutamine, and 1.5× vitamins (Life Technologies, Carlsbad, Calif.). The medium was supplemented with 10% fetal bovine serum (heat inactivated for 30 minutes in a 56° C. water bath), 2 mM L-glutamine, and 20 mg/L gentamycin. Cells were grown to subconfluency in T-25 flasks under 5% carbon dioxide at 37° C. in a Hereaus CO₂ incubator. The medium was changed before treatment to DMEM/F12 medium with 1.0 mg/mL human albumin (OSU Hospital Pharmacy, Columbus, Ohio), 5.0 mg/L human transferrin, and 5.0 mg/L bovine insulin.

Cells in T-25 flasks were treated with samples or 0.1% DMSO (negative control) or 10 nM letrozole (positive control) [in triplicate]. After 24 hours, the medium was changed, 50 nM androstenedione with 2 μCi [1β-³H]androst-4-ene-3,17-dione was added, and cells were incubated for 6 hours. The reaction mixture was then removed followed by precipitation of proteins using 10% trichloroacetic acid at 42° C. for 20 minutes. The mixture was centrifuged briefly and the aqueous layer extracted three times with CHCl₃ to remove unused substrate. The aqueous layer was treated subsequently with 1% dextran-coated charcoal. An aliquot of the aqueous layer was added to 3a70B scintillation cocktail for quantitation of the formation of ³H₂O. Results were corrected for blanks and for the amount of cells in each flask, determined by trypsinizing cells and analyzed using the diphenylamine DNA assay adapted to a 96-well plate format (Natarajan et al., 1994; Richards and Brueggemeier, 2003). Results are expressed as picomoles of ³H₂O formed per hour incubation per million live cells (pmol/h/10⁶ cells).

### Example 10

### Cell Viability Analysis

The effect of extracts and compounds on SK-BR-3 cell viability was assessed using a 3-(4,5-dimethylthiazol-2-yl)-2,5-diphenyl-2H-tetrazolium (MTT) bromide assay in six replicates. Cells were grown in custom media in 96-well, flat-bottomed plates for 24 h, and were exposed to various concentrations of extracts or compounds dissolved in DMSO (final concentration ≦0.1%) in define media for different time intervals. Controls received DMSO vehicle at a concentration equal to that in drug-treated cells. The medium was removed, replaced by 200 μl of 0.5 mg/ml of 3-(4,5-dimethylthiazol-2-yl)-2,5-diphenyl-2H-tetrazolium bromide in fresh media, and cells were incubated in the CO₂ incubator at 37° C. for 2 h. Supernatants were removed from the wells, and the reduced 3-(4,5-dimethylthiazol-2-yl)-2,5-diphenyl-2H-tetrazolium bromide dye was solubilized in 200 μl/well DMSO. Absorbance at 570 nm was determined on a plate reader.

While the compositions and methods have been described with reference to various embodiments, those skilled in the art will understand that various changes may be made and equivalents may be substituted for elements thereof without departing from the scope and essence of the disclosure. In addition, many modifications may be made to adapt a particular situation or material to the teachings of the disclosure without departing from the essential scope thereof. Therefore, it is intended that the disclosure not be limited to the particular embodiments disclosed, but that the disclosure will include all embodiments falling within the scope of the appended claims. In this application all units are in the metric system and all amounts and percentages are by weight, unless otherwise expressly indicated. All terms not specifically defined herein are considered to be defined according to Dorland's Illustrated Medical Dictionary, 27th edition, or if not defined in Dorland's dictionary then in Webster's New Twentieth Century Dictionary Unabridged, Second Edition. The disclosures of all of the citations, including patents and patent applications provided are being expressly incorporated herein by reference. The disclosed invention advances the state of the art and its many advantages include those described and claimed.

What is claimed:

1. A method of inhibiting aromatase activity comprising providing a composition of matter consisting essentially of an extract of mangosteen therapeutically effective for inhibiting aromatase activity.

2. A method of inhibiting aromatase activity comprising a providing a xanthone compound with aromatase inhibiting activity represented by Formula I: 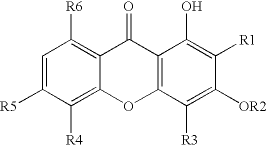
wherein:
R1 is a prenyl group or a hydrocarbon of five or more carbons or esters thereof;
R2 is —H, —OH, —CH3 or a hydrocarbon or esters thereof;
R3 is —H, a prenyl group, or a hydrocarbon of five or more carbons or esters thereof;
R4 is —H, —OH, —OCH3, a prenyl group or a hydrocarbon of five or more carbons or esters thereof; and
R5 is —H, or —OH;
R6 is —H, —OH, —OCH3, a prenyl group, a hydrocarbon of five or more carbons, a hydroxlyated hydrocarbon of 5 carbons or more, or esters thereof; and pharmaceutically acceptable salts thereof.

3. The method of claim 2 wherein R1 is a prenyl group, R2 is an —H, R3 is an —H, R4 is an —H, R5 is an —OH, and R6 is a prenyl group or a 5 carbon hydroxylated group.

4. The method of claim 3 wherein the compound is one or more of garcinone D and garcinone E, 1-isomangostin, mangostinone, α-mangostin, and γ-mangostin.

5. The method of claim 2 wherein said the compound is administered to a subject as a foodstuff, dietary supplement or pharmaceutical composition fortified with a xanthone according to Compound 1 or analog thereof having a therapeutically effective amount of activity in modulating undesired signal transduction activity useful for reducing the frequency, duration or severity of a disease or condition in a subject.

6. The method of claim 2 wherein the compound is provided to a subject who has, or is at elevated risk for acquiring a malignancy.

7. The method of claim 2 wherein the subject has, has had, or is at elevated risk of developing breast cancer or other estrogen sensitive disease.

8. A method of standardizing a nutraceutical product comprising identifying a xanthone from mangosteen with significant aromatase inhibiting ability to function as a marker compound; measuring the amount of said xanthone in the ingredients for said nutraceutical product; and adjusting the composition of said nutraceutical product by the addition of a given amount of said xanthone or inert ingredient wherein the standardized a nutraceutical product contains an identified concentration of said xanthone.

9. The method of claim 8 wherein the nutraceutical product is standardized to provide a given amount per dose of xanthone of one or more of cudraxanthone G, 8-deoxygartanin, garcinone D, garcinone E, gartanin, 8-hydroxycudraxanthone G, 1-isomangostin, α-mangostin, γ-mangostin, mangostinone, smeathxanthone A, and tovophylline A.

10. The method of claim 9 wherein the xanthone is one or more of garcinone D, garcinone E, α-mangostin, and γ-mangostin.

11. A nutraceutical product standardized according to claim 9.

12. A composition comprising an extract having a therapeutically effective amount of activity in modulating undesired signal transduction activity useful for reducing the frequency, duration or severity of a neoplastic disease or condition in a subject, said extract being derived from a plant of the genus Garcinia.

13. The composition of claim 12, wherein said disease or condition is selected from the group consisting of a malignancy, a neoplasia, an inflammatory disease or condition, an immunological disease, or aging.

14. The composition of claim 13, wherein the neoplasia is breast cancer.

15. The composition of claim 12, wherein the extract is obtained from the pericarp of mangosteen.

16. The composition of claim 12, wherein said amount of activity useful for modulating undesired signal transduction activity is present in an amount at least about 100% greater than present in the juice of mangosteen pericarp.

17. The composition of claim 12, in a form suitable for use in one or more of a foodstuff, a dietary supplement, a drug and a pharmaceutical composition, along with suitable carriers therefore.

18. A method for treating or preventing a disease or condition in a subject comprising the step of administering to said subject a therapeutically-effective amount of a foodstuff, dietary supplement or pharmaceutical composition fortified with a xanthone according to Compound 1 or analog thereof having a therapeutically effective amount of activity in modulating undesired signal transduction activity useful for reducing the frequency, duration or severity of a disease or condition in a subject.

19. The method of claim 18, wherein said disease or condition is selected from the group consisting of a malignancy, an immunological disease, or aging.

20. The method of claim 19, wherein the malignancy is an breast cancer.

21. The method of claim 18, wherein the xanthone is provided to a subject who has, or is at elevated risk for acquiring a malignancy.

22. The method of claim 18 wherein the xanthone is one or more of cudraxanthone G, 8-deoxygartanin, garcinone D, garcinone E, gartanin, 8-hydroxycudraxanthone G, 1-isomangostin, α-mangostin, γ-mangostin, mangostinone, smeathxanthone A, and tovophylline A.
